SEQUENCE LISTING (2 sequences)

SEQ ID NO: 1 - DNA, 1446 bp, synthetic construct
  source 1..1446: mol_type = other DNA
cgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggta

SEQ ID NO: 2 - DNA, 12 bp, synthetic construct
  source 1..12: mol_type = other DNA
cgcgaattcgcg

(19) United States
(12) Patent Application Publication
(10) Pub. No.: US 2023/0420045 A1
(43) Pub. Date: Dec. 28, 2023

(54) DNA-Based Data Storage Systems

(71) Applicant: The Board of Trustees of the University of Illinois, Urbana, IL (US)

(72) Inventors: Olgica Milenkovic, Urbana, IL (US); Charles M. Schroeder, Urbana, IL (US); Seyedkasra Tabatabaei, Urbana, IL (US); Aleksei Aksimentiev, Urbana, IL (US); Alvaro Gonzalo Hernandez, Champaign, IL (US); Chao Pan, Urbana, IL (US); Jingqian Liu, Urbana, IL (US); Shubham Chandak, Mountain View, CA (US); Bach Pham, Hanoi (VN); Min Chen, Amherst, MA (US); Spencer A. Shorkey, Montague, MA (US)

(21) Appl. No.: 18/171,764

(22) Filed: Feb. 21, 2023

Related U.S. Application Data
(60) Provisional application No. 63/312,334, filed on Feb. 21, 2022.

(51) Int. Cl. G11C 13/02; G16B 50/30; G16B 40/00; G16B 30/00; C12Q 1/6869

(52) U.S. Cl. CPC G11C 13/02; G16B 50/30; G16B 40/00; G16B 30/00; C12Q 1/6869

(57) ABSTRACT

The present disclosure relates generally to data storage using DNA sequences comprising synthetic nucleotides. In particular, the disclosure provides for a DNA data storage system comprising a covalently linked sequence of nucleotides, wherein the sequence of nucleotides comprises a modification region, wherein the nucleotides comprise synthetic nucleotides.

### CROSS-REFERENCE TO RELATED APPLICATIONS

This application claims the benefit of priority of U.S. Provisional Application No. 63/312,334, filed Feb. 21, 2022, and incorporated herein by reference in its entirety.

### GOVERNMENT RIGHTS

This invention was made with government support under 1618366, 1807526, and 200815 awarded by NSF. The government has certain rights in the invention.

### REFERENCE TO A SEQUENCE LISTING

The instant application contains a Sequence Listing which has been submitted electronically as a text file in ASCII format and is hereby incorporated by reference in its entirety. The Sequence Listing was created on Feb. 20, 2023, is named “22-0232-US_SequenceListing.xml” and is 4 kilobytes in size.

### FIELD

The present disclosure relates to DNA-based data storage systems, and methods of preparing, using and reading the same.

### BACKGROUND

DNA is emerging as a robust data storage medium that offers ultrahigh storage densities greatly exceeding conventional magnetic and optical recorders. Information stored in DNA can be copied in a massively parallel manner and selectively retrieved via polymerase chain reaction (PCR). However, existing DNA storage systems suffer from high latency caused by the inherently sequential writing process. Despite recent progress, a typical cycle time of solid-phase DNA synthesis is on the order of minutes, which limits the practical applications of this molecular storage platform. Using current technologies, writing 100 bits of information (or, roughly two words) requires nearly two hours and costs more than US$1, assuming that each nucleotide stores its theoretical maximum of two bits. To overcome these challenges, new synthesis methods and information encoding approaches are required to accelerate the speed of writing large-volume data sets (Fan J, Han F, Liu H. Challenges of Big Data analysis. National Science Review. 2014 Jun. 1; 1(2):293-314).

Expanding the alphabet of a DNA storage media by including chemically modified DNA nucleotides can both increase the storage density and the writing speed because more than two bits are recorded during each synthesis cycle. However, designing chemically modified nucleotides as new letters for the DNA storage alphabet must be tightly coupled to the process of reading the encoded information via DNA sequencing, because current DNA sequencing methods, including single-molecule nanopore sequencing, have been developed and optimized to read natural nucleotides. Prior work reported an expanded nucleic acid alphabet of synthetic DNA and RNA nucleotides that can be replicated and transcribed using biological enzymes (Hoshika S, Leal N A, Kim M-J, Kim M-S, Karalkar N B, Kim H-J, et al. Hachimoji DNA and RNA: A genetic system with eight building blocks. Science. 2019 Feb. 22; 363(6429):884-7), but this alphabet was not designed for molecular storage applications and was not accurately read using a nucleic acid sequencing method. Aerolysin nanopores were used to detect synthetic polymers flanked by adenosines, where each monomer of the polymer carries one bit of information (Cao C, Krapp L F, Al Ouahabi A, Konig N F, Cirauqui N, Radenovic A, et al. Aerolysin nanopores decode digital information stored in tailored macromolecular analytes. Sci Adv. 2020 December; 6(50): eabc2661). Recently, it was reported that a base pair containing a single chemically modified nucleotide can be detected using biological nanopores (Ledbetter M P, Craig J M, Karadeema R J, Noakes M T, Kim H C, Abell S J, et al. Nanopore Sequencing of an Expanded Genetic Alphabet Reveals High-Fidelity Replication of a Predominantly Hydrophobic Unnatural Base Pair. J Am Chem Soc. 2020 Feb. 5; 142(5):2110-4). Despite recent advances, single-molecule detection and sequencing of an expanded molecular alphabet based on a library of chemically diverse modified nucleotides has not yet been demonstrated.

Accordingly, there remains a need to develop new DNA-based storage system along with efficient and high fidelity methods of decoding.

### SUMMARY

The present disclosure concerns DNA-based storage systems incorporating synthetic DNA nucleotides. This approach allows high-density information storage. Further, methods of accurately reading novel sequence comprised of mixtures of synthetic and natural DNA are demonstrated.

Accordingly, one aspect of the present disclosure is DNA data storage systems comprising a covalently linked sequence of nucleotides, wherein the sequence of nucleotides comprises a modification region, wherein the nucleotides comprise synthetic nucleotides.

In another aspect, the present disclosure provides for methods of reading a DNA sequence, the method comprising:
introducing a DNA data storage system into a flow cell of a nanopore sequencing device, wherein the DNA data storage system comprises a modification region comprising synthetic nucleotides;
receiving information indicative of an electrical signal provided when the modification region passes through a nanopore of the nanopore sequencing device;
classifying, based on the received information, at least a portion of the modification region according to an expanded molecular alphabet; and
determining, based on the classifying, a nucleotide sequence of the modification region.

In another aspect, the present disclosure provides for methods of training a neural network comprising:
providing training data to the neural network, wherein the training data comprises labeled data, wherein the labeled data comprises values indicative of electrical signals provided when a modification region of a DNA data storage system passes through a nanopore of a nanopore sequencing device, wherein the labeled data further comprises labels corresponding to an expanded molecular alphabet; and
comparing an output of the neural network to the labels;
adjusting at least one weight of the neural network based on the comparison.

Other aspects of the disclosure will be apparent to those skilled in the art in view of the description that follows.

### BRIEF DESCRIPTION OF THE DRAWINGS

FIG. 1: DNA data storage using natural and chemically modified nucleotides. (A) Chemical structures of natural DNA nucleotides (A, C, G, T) and the selected chemically modified nucleotides employed in our study (B1-B7). (B) Schematic of the ssDNA oligo used in MspA nanopore experiments. The length of the oligos is 40 nucleotides (nts), with biotin attached at the 5′ terminus. Homo- or heterotetrameric sequences are located at positions 13-16, flanked by two polyT regions of length 12 nt and 24 nt on the 5′ and 3′ ends, respectively. (C) Sequence space for DNA homotetramers or heterotetramers used in MspA nanopore experiments. The notation aX+bY, where a and b take values in {2, 3, 4} so that a+b=4, indicates that ‘a’ symbols of the same kind are combined with ‘b’ symbols of another kind and arranged in an arbitrary linear order. In total, 77 distinct tetrameric sequences were synthesized and tested experimentally. (Left) Circular diagram showing all 11 homotetramers and 12 tetrameric sequences of the form ACT+X, where X is a chemically modified nucleotide from the set {B2, B3, B5}. (Middle) Circular diagram showing all 30 tested combinations of tetrameric sequences with total composition 2X+2Y using chemically modified monomers from the set {B1, B2, B3, B4, B5}, including sequence patterns XXYY, XYYX, and XYXY. (Right) Circular diagram showing the remaining 24 combinations of tetrameric sequences with total composition 3X+Y using the set {B2, B3, B5}. Five chemically modified nucleotides form stable base pairs with natural nucleotides via hydrogen bonds (B2 G, B3-A, B5-A, B6-A, B6-C), based on the results from molecular dynamic (MD) simulations.

FIG. 2: Identification of chemically modified DNA using MspA nanopores. (A) Schematic diagram of ssDNA immobilized in a MspA nanopore, where ssDNA containing a biotin-streptavidin interaction at the 5′ terminus prevents translocation through the pore. Residual ion current generated by four nucleotides at positions 13-16 from the 5′ terminus is recorded for ssDNA immobilized in the pore. (B) Histograms of average residual ionic currents Iᵣₑₛ shown in gray for different homopolymers (A, T, C, G, and B1-B7). The fitted Gaussian curves are depicted in red for natural nucleotides (A, T, C, G), and in blue for chemically modified nucleotides (B1-B7). (C) Histograms of the average residual ionic currents and the fitted Gaussian curves at various applied voltages for tetramers involving different combinations and orderings of B2 and B3. (D) Peak values (points) and confidence intervals (bars) of the fitted Gaussians with mean residual ionic currents corresponding to tetramers obtained by inserting one of the monomers B2 and B3 into the sequence ACT, at applied biases of 150 mV and 180 mV. (E) Schematic of the shift reconciliation method for resolving ambiguities in the readouts of different tetramers.

FIG. 3: Sequencing oligos containing chemically modified nucleotides using ONT GridION. (A) Schematic of oligo design and a picture of the GridION sequencer used in our experiments. (B)(Left) Illustration of current levels of polyA and polyT regions, used in our custom level-calibration scheme. Dashed orange circle indicates the region harboring the signals from chemically modified nucleotides. (Right) Region-of-interest in raw current signal obtained by identifying polyA-polyT patterns. (C) Neural network model used for classification. The 1D residual neural network architecture comprises nine 1D convolution blocks. For example, a 1D convolution block (1×8 conv,64) indicates that the kernel size for the convolution is 1×8 and that the number of output channels is 64. Half-downsampling for each channel is denoted by (/2); averaging over all channels to arrive at a single vector is referred to as “Average Pooling”; the (fc 128×30) notation indicates a fully connected layer with the shape 128×30. (Right) Magnified view of the operation of 1D convolutional neural networks on time-series data. (D) (Top) Confusion matrix for 66 classes, all of which have roughly the same number of samples (subsampled to ˜3500 sample oligos in each class). Random guessing would lead to a classification accuracy of 1.52%, whereas the smallest accuracy from our model is 41% (tetramer 2252). For our model-based prediction, the mean classification accuracy is 60.28%±0.28% (39× larger than random guessing), and the highest observed accuracy is 79% (tetramer 1111). The exact number of samples in each class is listed in Table 5. (Bottom left) Confusion matrix for six selected classes using B2 and B4 (named as listed, subsampled to roughly 5000 samples per class). Random guessing leads to an accuracy of 16.67%, whereas our model-based prediction ensures an average classification accuracy of 72.25%±1.46%. (Bottom right) Confusion matrix for six selected classes using B4 and B5 (named as listed, subsampled to roughly 5000 samples per class). Random guessing leads to an accuracy of 16.67%, while our model-based prediction ensures an average accuracy of 77.84%±0.96%.

FIG. 4: Stability of DNA duplexes containing chemically modified nucleotides. The backbone of the dodecamer is shown using silver spheres whereas the bases are drawn as molecular bonds. Chemically modified bases and the natural bases that pair with them are colored according to the atom type (cyan for carbon, blue for nitrogen and red for oxygen). Base pairs immediately adjacent to the modified base pair are colored in red or blue. (A) Microscopic configurations of modified base pairs (from top to bottom: B2-G, A-B3, A-B5, A-B6 and C-B6). (B) Donor (N1)-acceptor (N3) distance (black) in the modified base pair (black) and in the adjacent base pairs (red and blue) during the last 100 ns of the 350 ns MD simulation. The arrows indicate the correspondence between the base pairs and the curves. The curves show a running average of the 10 ps-sampled data with a 2 ns averaging window. (C) Microscopic configuration of modified base pairs. The black lines represent hydrogen bonds. The donor and the acceptor are labeled asides the atoms. (D) Probability of observing the specified number of hydrogen bonds within a modified base pair. The H-bonding probabilities were computed using the final 100 ns of a 350 ns all-atom MD simulation of a DNA dodecamer.

FIG. 5. Discrimination of immobilized DNA by MspA nanpore. (A) Schematic diagram of DNA immobilized in the MspA nanopore. Singe-stranded DNA (ssDNA) was attached to a streptavidin molecule (cyan) using a biotin linker. Bulky streptavidin prevents ssDNA to translocate through the MspA pore (gray). The residual ion current was recorded as the ssDNA is immobilized within the pore, which is generated by 4 nucleotides in and around the constriction side, at positions 13-16 from the biotin-streptavidin end. The open-pore current of MspA is normalized to 100%. (B) The representative single-channel recording generated by each tetramer sequence at positions 13-16 from the tethering point to the constriction site (reading head) of the MspA pore. Native nucleotides are highlighted in blue and modified nucleotides in red. Buffer used is 1 M KCl 10 mM HEPES pH 8.0.

FIG. 6. Histograms of the averaged residual ionic currents and the fitted Gaussian curves at various applied voltages for tetramers involving different orderings of B2 and B5 monomers (A) and B3 and B5 monomers (B) at 150, 180, and 200 mV. All experiments were performed in aqueous buffer (1 M KCl 10 mM HEPES pH 8.0). (C) Peak values and full-width half-height values (FWHM), represented as error bars, of the fitted Gaussian distributions around mean residual ionic currents generated by different orderings of B5 with the natural nucleotides (A, C, and T) at 150,180, and 200 mV. All experiments were performed in aqueous buffer (1 M KCl 10 mM HEPES pH 8.0).

FIG. 7. (A) (Left) Raw current readout of a control oligo bearing the content CCCC. (Right) A raw current readout bearing the content 2233. The red and green lines represent the expected standard levels for polyA and polyT regions, respectively. (B) Analysis of nanopore sequencing results for chemically modified nucleotides. (Top Left) Raw current readout for a control oligo containing the sequence 2233. (Top Right) Visualization of the kernel density estimation method: Two peaks correspond to two possible polyA region levels. (Bottom) The procedure for determining which level to use for calibration, based on the mean value of the “nearly-flat” region following the predicted polyA region. An example of the current level corresponding to the highest peak, which was used to correctly estimate the location of the polyA region. Building upon this step, the results show that one can also isolate the signal region which corresponds to the chemically modified nucleotides.

FIG. 8. Classification performance of 12 different classes of tetramers. The names of the classes are listed in the subfigures, along with their average classification accuracies: (1) 69.39±0.93%, (2) 72.25%±1.46%, (3) 68.87%±0.90%, (4) 77.84%±0.96%, (5) 72.18%±1.79%, (6) 71.97%±0.54%, (7) 81.27%±0.93%, (8) 79.17%±1.87%, (9) 69.66%±0.48%, (10) 80.04% 0.69%, (11) 70.81%±1.15%, (12) 88.00% 1.31%.

FIG. 9. Interactions between modified and natural bases that do not involve stable hydrogen bonds. (A) Microscopic configurations of modified base pairs (from top to bottom: B1-T, B2-G, A-B4, G-B4, C-B4, T-B4, G-B6, and T-B6). The backbone of the dodecamer is shown using silver spheres whereas the bases are drawn as molecular bonds. Unnatural bases and the natural bases that pair with them are colored according to the atom type (cyan for carbon, blue for nitrogen and red for oxygen). Base pairs immediately adjacent to the modified base pair are colored in red or blue. (B) Distance between the key atoms of the modified base pair during the last 100 ns of the 350 ns MD simulation. The red curve and blue curve show the N1-N3 distance for the two adjacent base pairs, whose pairing patterns can either remain intact or be disrupted. The arrows starting from panel A to panel B indicate the correspondence between the base pairs and the curves. The label specifies the atoms used to compute the distance. The curves show a running average of the 10 ps-sampled data with a 2 ns averaging window. (C) Probability of observing the specified number of hydrogen bonds within a modified base pair. The H-bonding probabilities were computed using the final 100 ns of a 350 ns all-atom MD simulation of a DNA dodecamer. (D) As a starting point to experimentally evaluate the effect of chemically modified nucleotides on DNA structure, a PCR reaction was performed on a 1.4 kb double stranded DNA from a commonly used vector, pUC19 plasmid, using Q5 polymerase. The reaction was either supplied by all four natural nucleotides or B1 and B2 as substitutes for A and C. The final PCR products were run on 1% agarose gel. The results indicate successful incorporation of B1 and B2 into DNA duplex structure when only one of them (lanes 2 and 3) or two of them (lane 4) were used instead of the natural nucleotides. (E) Initial state of a simulation system where a DNA dodecamer containing chemically modified nucleotides is immersed in electrolyte solution. The backbone of the dodecamer is shown using silver spheres whereas the bases are drawn as molecular bonds. Chemically modified bases and the natural bases that pair with them are colored according to the atom type (cyan for carbon, blue for nitrogen and red for oxygen). Base pairs immediately adjacent to the modified base pair are colored in red or blue.

FIG. 10: Interactions between B4 and natural bases in long DNA strands (A) Microscopic configurations of modified base pairs (from top to bottom: A-B4, G-B4, C-B4 and T-B4). The backbone of the dodecamer is shown using silver spheres whereas the bases are drawn as molecular bonds. B4 bases and the natural bases that pair with them are colored according to the atom type (cyan for carbon, blue for nitrogen and red for oxygen). Base pairs immediately adjacent to the modified base pair are colored in red or blue. In contrast to simulations reported in FIG. 8, here each DNA dodecamer contains only one B4 base. Extra bonds between donor (N1) and acceptor (N3) (The equilibrium length was set as 2.9 Å. The spring constant was set as 1 kcal/mol/Å²) are applied the terminal base pairs, preventing DNA from fraying and thereby mimicking an environment of a longer DNA strand. (B) Distance between the key atoms of the modified base pair during the last 50/100 ns of the MD simulation. The red curve and blue curve show the N1-N3 distance for the two adjacent base pairs, whose pairing patterns can either remain intact or be disrupted. The arrows starting from panel A to panel B indicate the correspondence between the base pairs and the curves. The label specifies the atoms used to compute the distance. The curves show a running average of the 10 ps-sampled data with a 2 ns averaging window. (C) Probability of observing the specified number of hydrogen bonds within a modified base pair. The H-bonding probabilities were computed using the final 50/100 ns of the all-atom MD simulations of a DNA dodecamer.

### DETAILED DESCRIPTION

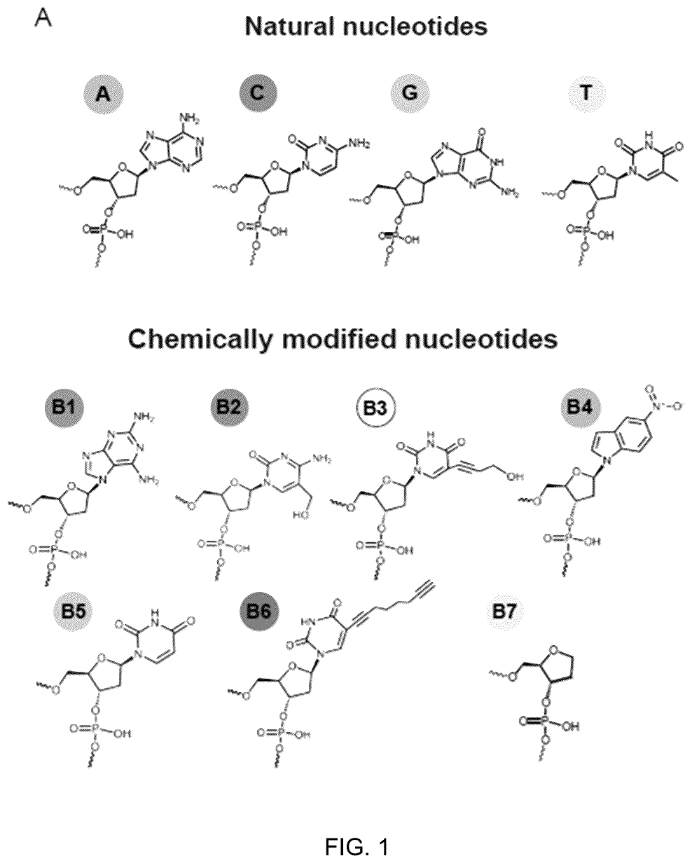

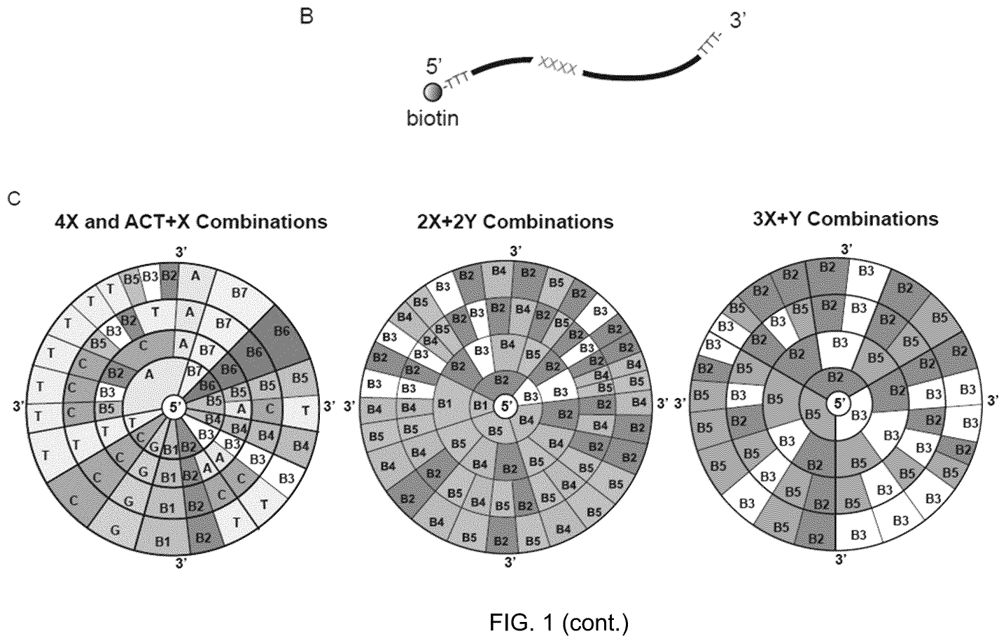

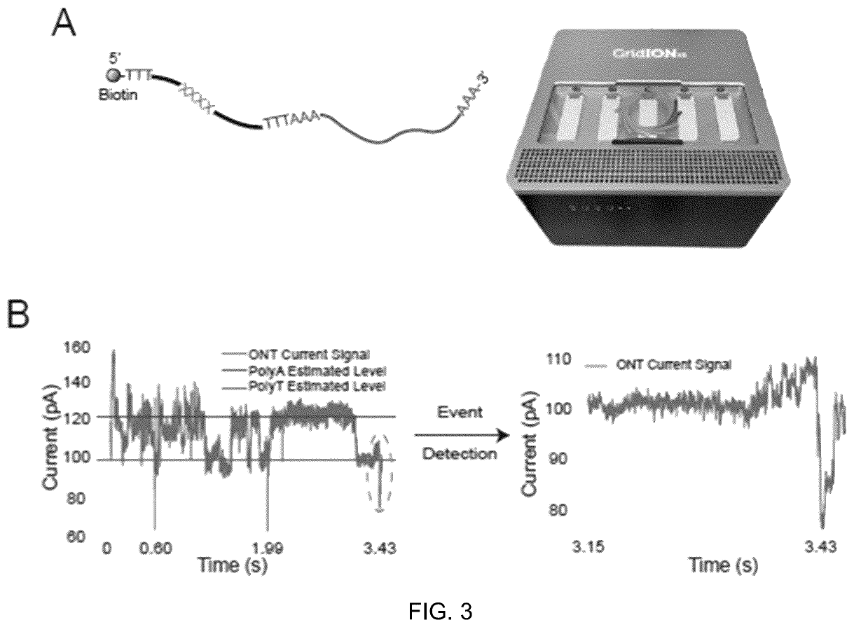

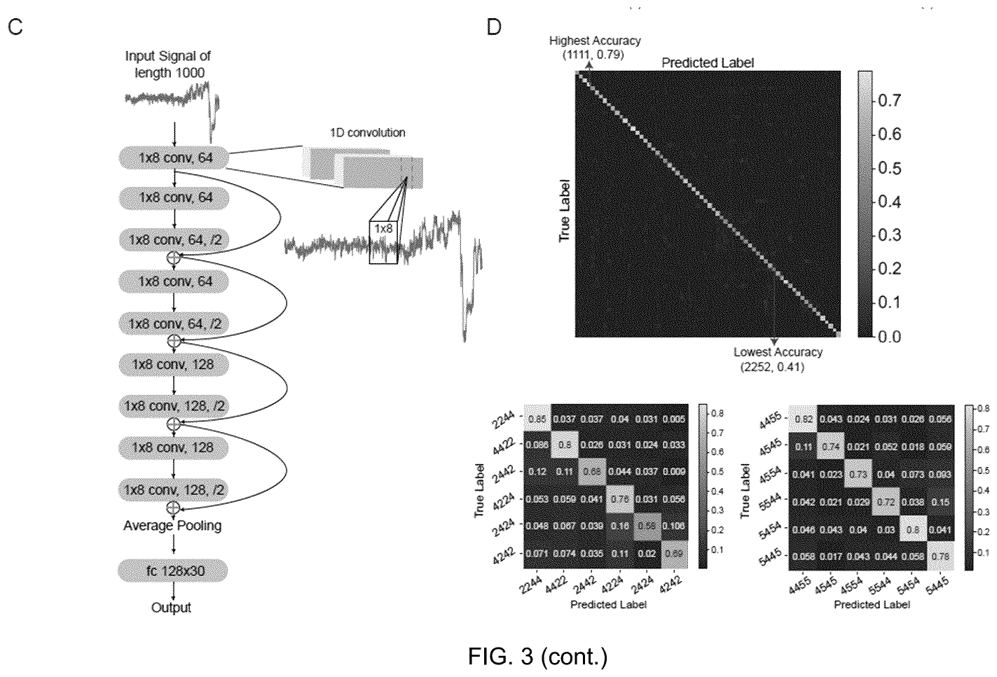

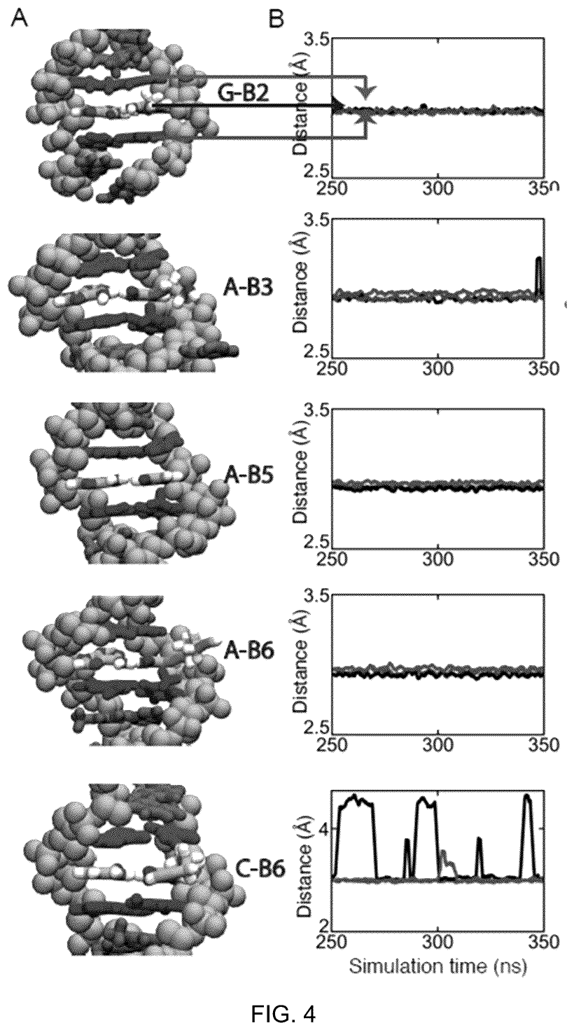

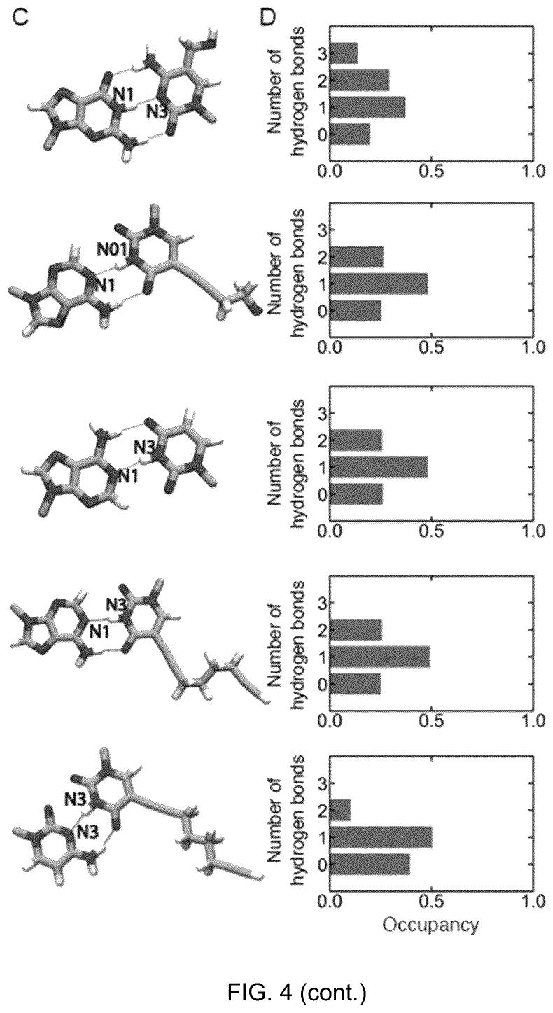

Here, an expanded molecular alphabet for DNA data storage comprising four natural and seven chemically modified nucleotides is disclosed that is readily detected and distinguished using nanopore sequencers (FIG. 1 and Table 1). Our results show that MspA nanopores can accurately discriminate 77 combinations and orderings of chemically diverse monomers within homo- and heterotetrameric sequences (FIGS. 1-2, 5-6, Tables 2-4). Highly accurate classification (exceeding 60% on average) of combinatorial patterns of natural and chemically modified nucleotides is possible using deep learning architectures that operate on raw current signals generated by GridION of Oxford Nanopore Technologies (ONT) (FIGS. 3 and 7-8). Furthermore, the stability of DNA duplexes containing modified nucleotides using all-atom molecular dynamics (MD) simulations has been described (FIGS. 4, 9-10 and Table 6). Overall, the extended molecular alphabet offers a nearly two-fold increase in storage density and potentially the same order of reduction in recording latency, thereby providing a promising path forward for the development of new molecular recorders.

Accordingly, one aspect of the present disclosure is DNA data storage systems comprising a covalently linked sequence of nucleotides, wherein the sequence of nucleotides comprises a modification region, wherein the nucleotides comprise synthetic nucleotides. For examples, in various embodiments as otherwise described herein, the synthetic nucleotides are each independently of the formula:

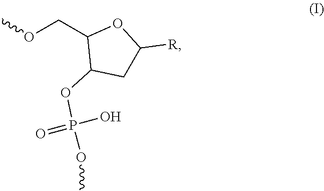

wherein R is H, or is a heterocycle. For example, in particular embodiments, wherein R is not H, R is capable of making at least one hydrogen bond to a natural nucleotide. Motifs that are suitable for hydrogen bonding to natural nucleotides are known in the art, and the skilled person would be able to ascertain, in light of the present disclosure, whether a particular group is capable of hydrogen bonding a natural nucleotide. For example, suitable hydrogen-bond forming groups include heterocycles comprising an electronegative element, such as N, O, or S.

As otherwise described herein, in various embodiments, synthetic nucleotides are those that are structurally distinct from natural nucleotides, such that they give a distinct signal when read according to methods as described herein.

In certain embodiments as otherwise described herein, R is H or a nitrogen-containing heterocycle, wherein the heterocycle is monocyclic or fused bicyclic (e.g., an optionally substituted heterocycle). For examples, in particular embodiments, R is H,

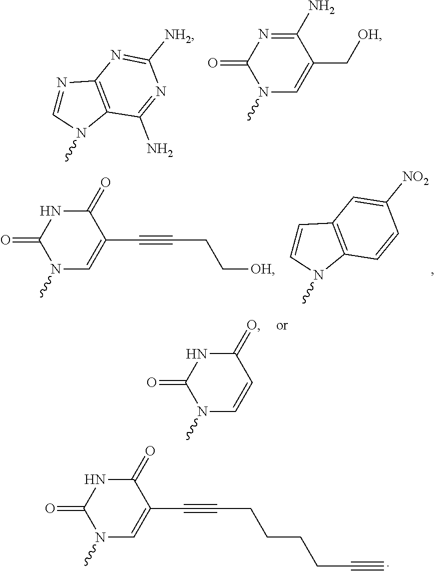

Unless otherwise indicated herein, the disclosed structures contemplate any suitable salts thereof. In various embodiments as otherwise described herein, the heterocycles as disclosed herein may be optionally substituted, e.g., substituted with 0-3 R groups. For example, in some embodiments, each R is halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —OC(O)NH₂, —OC(O)NH(C₁-C₁₀ alkyl), —OC(O)N(C₁-C₁₀ alkyl)₂, —CO₂H, —CO₂(C₁-C₁₀ alkyl), —CHO, —CO(C₁-C₁₀ alkyl), or —OC(O)(C₁-C₁₀ alkyl). As used herein, each alkyl group is optionally substituted with 1-5 R^{A} group, wherein each R^{A} is halogen, —NO₂, —CN, NH₂, —OH, —CO₂H, or —CONH₂.

Heterocycles as described herein may be heteroaromatic cycles or heterocycloalky moieties. The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl-N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl may have 1, 2, 3 or 4 heteroatoms. The heterocycloalkyl may be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2,5-diazabicyclo[2.2.1]heptyl, morpholinyl, thiomorpholinyl, thiomorpholinyl S-oxide, thiomorpholinyl S,S-dioxide, 2-oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S-oxide, tetrahydrothienyl S,S-dioxide and homothiomorpholinyl S-oxide. Especially desirable heterocycloalkyl groups include morpholinyl, 3,4-dihydroisoquinolin-2(1H)-yl, tetrahydropyranyl, piperidinyl, aza-bicyclo[2.2.2]octyl, γ-butyrolactonyl (i.e., an oxo-substituted tetrahydrofuranyl), γ-butryolactamyl (i.e., an oxo-substituted pyrrolidine), pyrrolidinyl, piperazinyl, azepanyl, azetidinyl, thiomorpholinyl, thiomorpholinyl S,S-dioxide, 2-oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “halo” or “halogen” as used herein, means —Cl, —Br, —I or —F. For example, in certain embodiments, halogen is —F.

In certain applications, the addition of bulky groups to the sequence of nucleotides may aid in their application, for example by preventing complete translocation through nanopores. Accordingly, in various embodiments as otherwise described herein, the sequence of nucleotides further comprises biotin, for example, a 5′-bound biotin. In particular embodiments, the sequence of nucleotides further comprises streptavidin bound to a 5′-bound biotin.

As described herein, calibration of the DNA sequence can be used to assist in data storage and recovery. Accordingly, in certain embodiments as otherwise described herein, the covalently linked sequence of nucleotides comprises a calibration region. For example, the calibration region may be a known sequence so that a known signal will be read in order to standardize or otherwise calibrate signal output. For example, in particular embodiments, the calibration region comprises a poly-A region.

As described herein, the DNA sequence may contain a plurality of synthetic nucleotides. In various embodiments, the synthetic nucleotides are of a variety of structures, and each structure may or may not be repeated, for example to encode information. Accordingly, in certain embodiments as otherwise described herein, the DNA data storage system comprises at least 2 and no more than 10 distinct synthetic nucleotides. For example, in some embodiments, the DNA data storage system comprises 2-8 distinct synthetic nucleotides, or 3-8 distinct synthetic nucleotides, or 4, 5, 6, or 7 distinct synthetic nucleotides. In various embodiments, the synthetic nucleotides may be provided in sequence with natural nucleotides, for example, wherein the modification region comprises both synthetic nucleotides and natural nucleotides.

In another aspect, the present disclosure provides for methods of reading a DNA sequence, the method comprising:
introducing a DNA data storage system into a flow cell of a nanopore sequencing device, wherein the DNA data storage system comprises a modification region comprising synthetic nucleotides;
receiving information indicative of an electrical signal provided when the modification region passes through a nanopore of the nanopore sequencing device;
classifying, based on the received information, at least a portion of the modification region according to an expanded molecular alphabet; and
determining, based on the classifying, a nucleotide sequence of the modification region.

As described herein, a neural network is a type of machine learning algorithm that can be modeled after the structure of the human brain. In such scenarios, the neural network may include a plurality of interconnected nodes or neurons that process information and communicate with each other. The neural network may include three main types of layers: input, hidden, and output. The input layer is where the data is initially fed into the network, the output layer produces the final output or prediction, and the hidden layer(s) are where the majority of the computation takes place. Each neuron in the network takes in inputs from other neurons, applies a mathematical function to these inputs, and produces an output that is sent to other neurons in the network.

Each neuron is associated with a set of weights, which are parameters that determine the strength and direction of the connections between neurons. When an input signal is received by a neuron, it is multiplied by the weights associated with that neuron, and the resulting value is passed through an activation function to produce the output of the neuron.

During a training process, the neural network adjusts the weights and biases of its neurons in order to minimize the difference between its predictions and the actual output. The training process may include a process called backpropagation, which involves propagating errors backwards through the network and adjusting the weights and biases accordingly.

In some examples, the neural network may be trained with training data. In such scenarios, training data could include a set of labeled examples that may teach a neural network how to make predictions or classifications. In some example embodiments, the data could include inputs and corresponding outputs, where the inputs represent the features or attributes of the data, and the outputs represent the desired outcome or label for each input. Additionally or alternatively, the neural network may be trained using unsupervised learning, where the training data consists of only the inputs, and the network learns to identify patterns and features in the data without explicit output labels.

In some embodiments, the neural network may include one or more convolutional layers. In such scenarios, the convolutional layer is a type of layer in a neural network that is designed to analyze data that has a grid-like structure, such as an image. The convolutional layer applies a set of filters, or kernels, to different parts of the input data, allowing the network to identify patterns and features in the data. In some embodiments, the filters in a convolutional layer are small matrices of weights that slide over the input data, performing element-wise multiplication and addition to produce a single output value for each location the filter is applied to. This process is known as a convolution operation. The resulting output of convolutional operation is called a feature map, which may contain information about the presence or absence of certain patterns or features in the input data.

Convolutional layers may be followed by pooling layers, which downsample the feature maps by taking the maximum or average value of a small region of the feature map, allowing the network to focus on the most important features while reducing the dimensionality of the data.

In some examples, the neural network may include one or more fully connected layers, also known as dense layers. A fully connected layer is a type of layer in a neural network where every neuron in the layer is connected to every neuron in the previous layer. In other words, the neurons in a fully connected layer receive input from all of the neurons in the previous layer.

The output of each neuron in a fully connected layer is calculated by taking a weighted sum of the inputs from the previous layer, and passing this sum through an activation function. The weights and biases associated with each neuron are learned during the training process, allowing the network to learn complex nonlinear relationships between the input and output.

In another aspect, the present disclosure provides for methods of training a neural network comprising:
providing training data to the neural network, wherein the training data comprises labeled data, wherein the labeled data comprises values indicative of electrical signals provided when a modification region of a DNA data storage system passes through a nanopore of a nanopore sequencing device, wherein the labeled data further comprises labels corresponding to an expanded molecular alphabet; and
comparing an output of the neural network to the labels;
adjusting at least one weight of the neural network based on the comparison.

In some embodiments, the neural network comprises a 1-dimensional residual neural network. In such scenarios, the 1-dimensional residual neural network could include:
a plurality of 1-dimensional convolution layers; and
a fully connected layer, wherein the adjusting further comprises adjusting at least one weight of at least one 1-dimensional convolution layer or at least one weight of the fully connected layer.

### EXAMPLES

The Examples that follow are illustrative of specific embodiments of the disclosure, and various uses thereof. They are set forth for explanatory purposes only, and should not be construed as limiting the scope of the disclosure in any way.

Results and Discussion

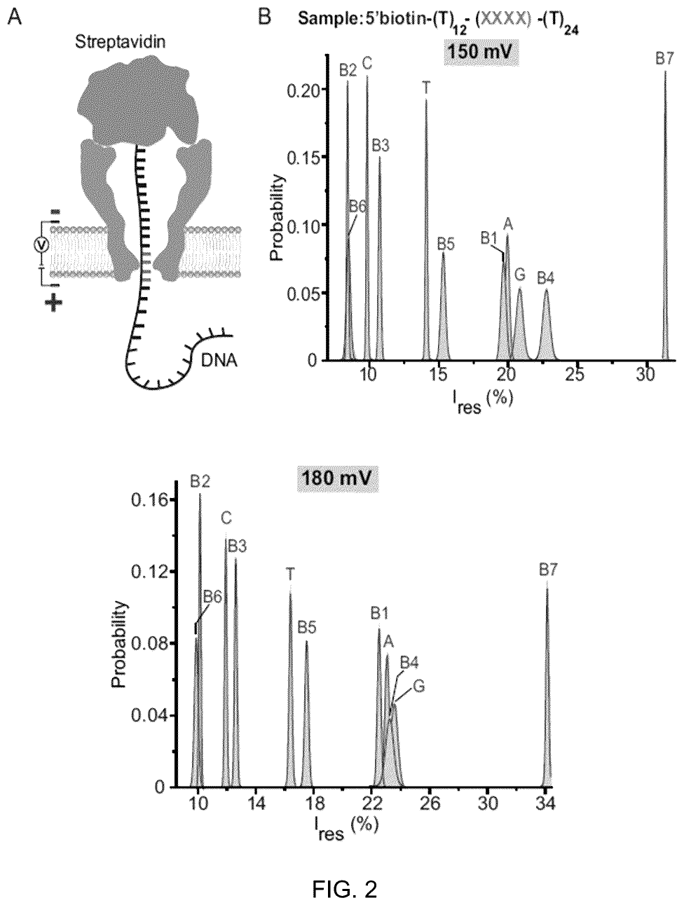

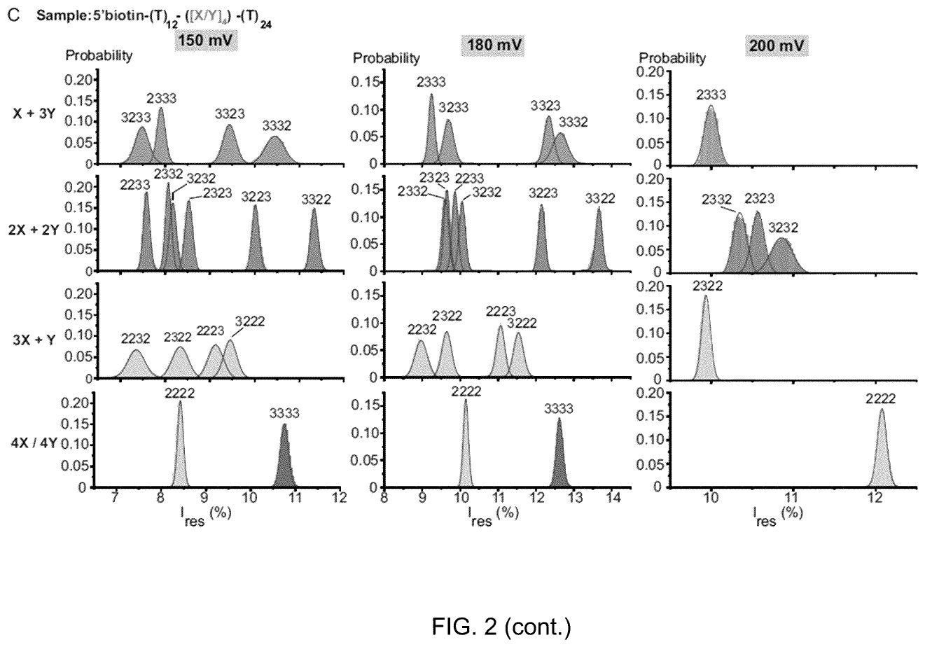

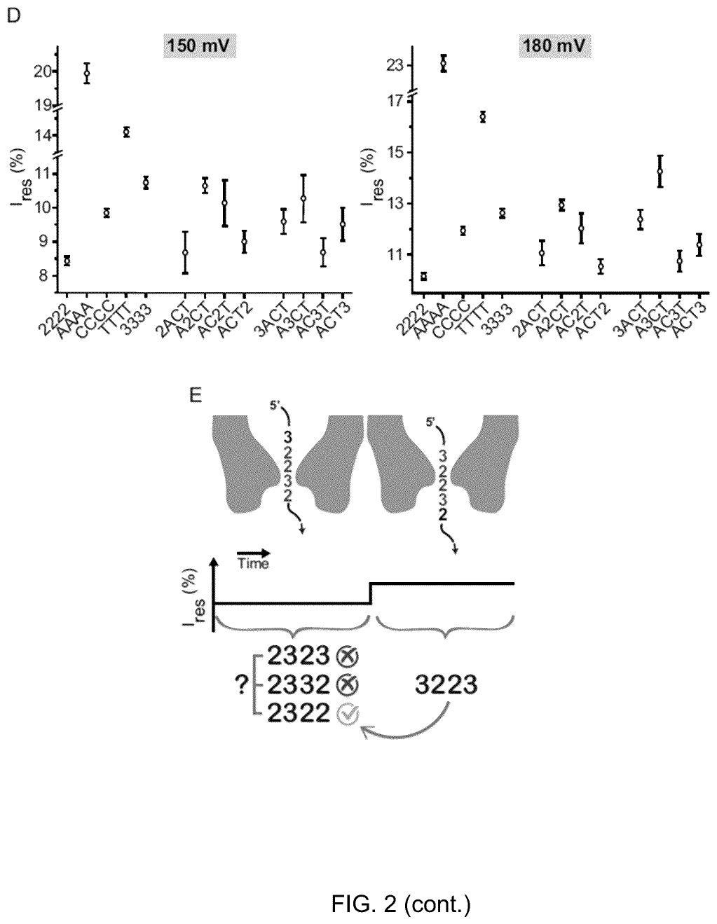

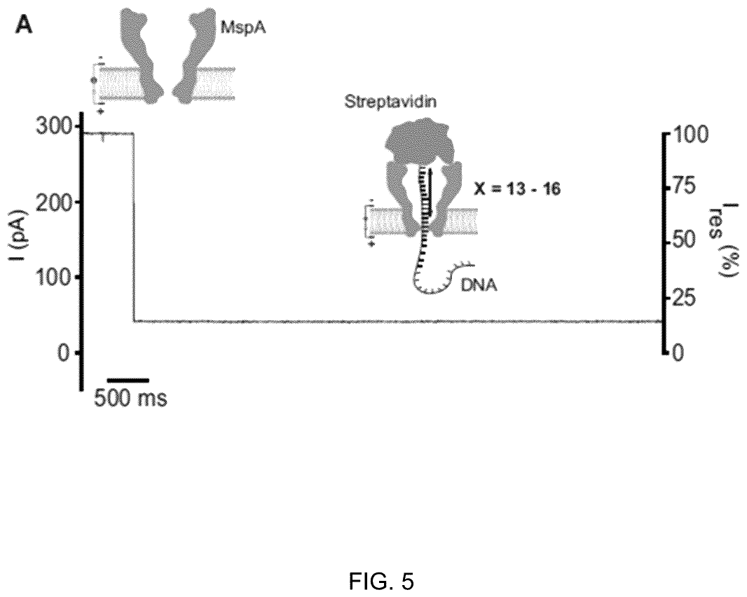

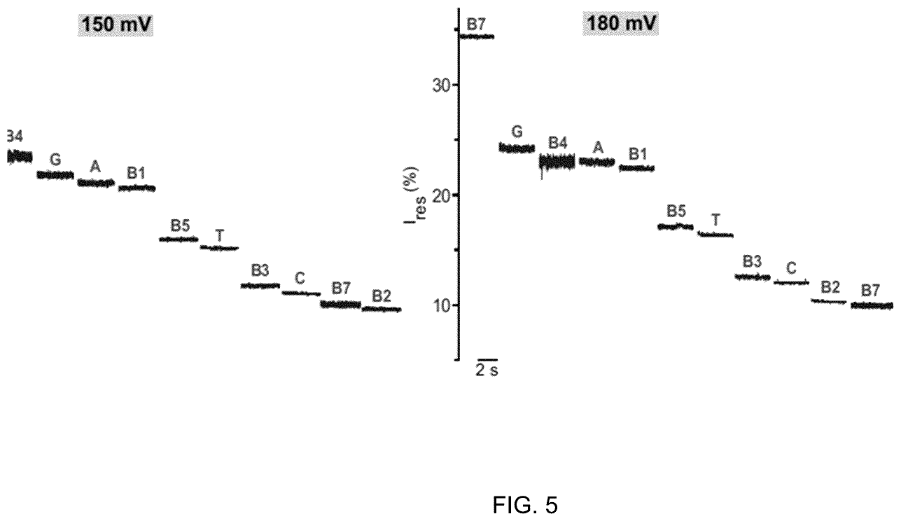

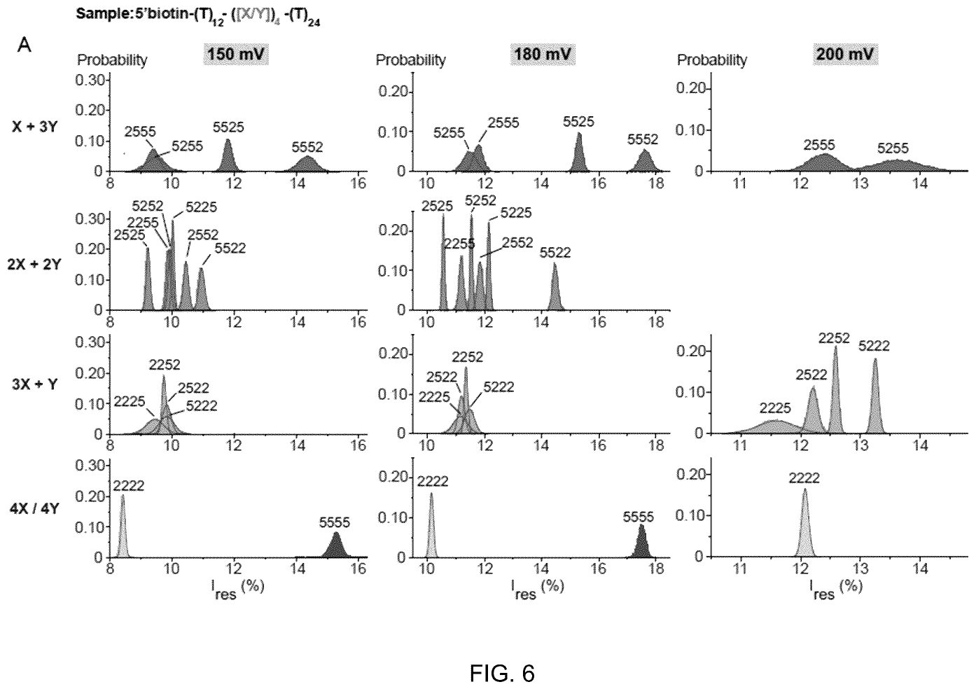

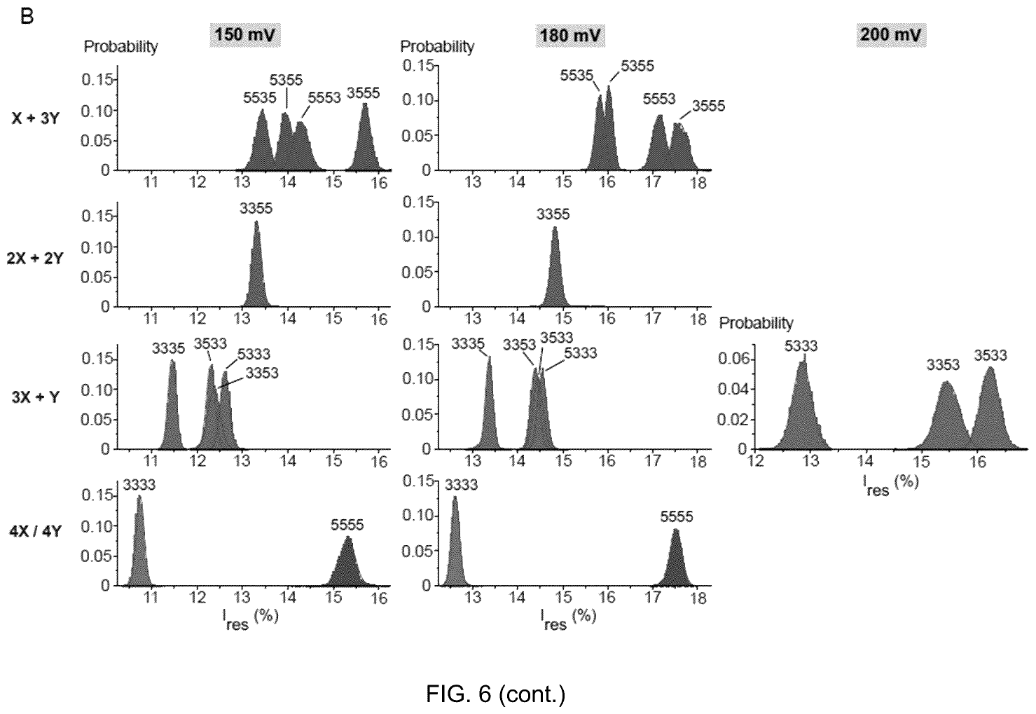

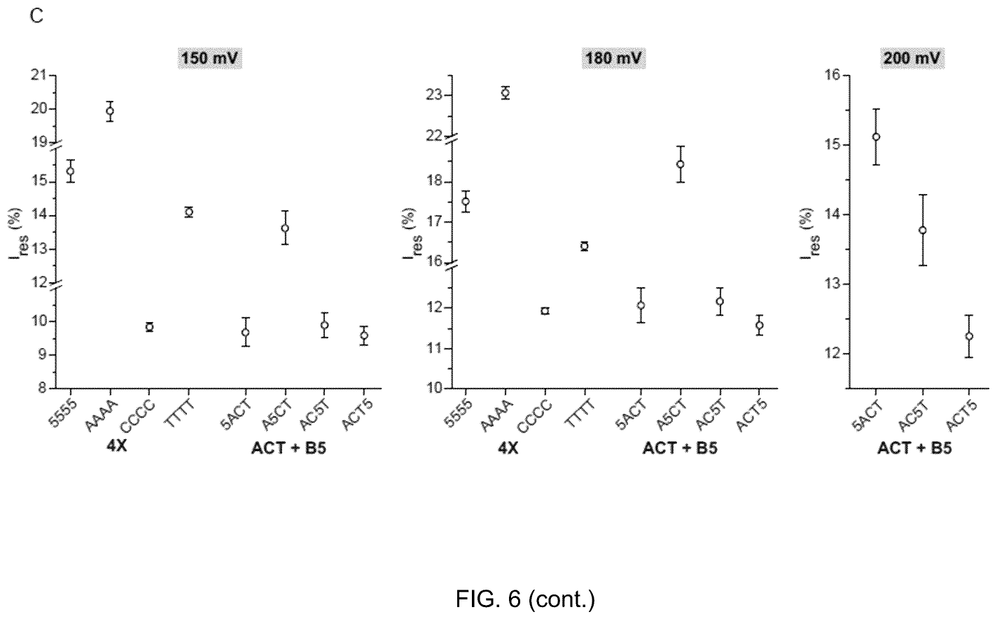

To determine whether natural and chemically modified DNA nucleotides can be distinguished using the biological nanopore MspA, a series of single-stranded DNA (ssDNA) molecules with the general sequence 5′-biotin-(dT)₁₂-XXXX-(dT)₂₄-3′, where X={A, T, C, G, B1-B7} was designed (FIG. 2, FIGS. 5-6, Tables 2-4). It has been hypothesized that specific chemical modifications to nucleobases such as amines, alkynes, or indole moieties can alter polymer-amino acid interactions in biological nanopores, thereby generating distinct signals in nanopore readouts. In the process, the stability of base pairing and base stacking interactions between natural and chemically modified nucleotides using a combination of MD simulations and experiments was also considered (Tables 1 and 6, FIGS. 4, 9-10). Stability is important for long term storage applications.

Following molecular design and synthesis of ssDNA oligos, MspA nanopore experiments were performed where ssDNA oligos containing streptavidin at the 5′ terminus were electrophoretically attracted inside MspA nanopores. The bulky streptavidin protein prevents the oligos from fully translocating through the pore without appreciably affecting the measured ionic currents. Consequently, ssDNA molecules are effectively immobilized within MspA nanopores, exposing the four nucleotides at positions 13-16 from the tethering point to the constriction of the MspA pore (FIG. 2A). In this assay, streptavidin holds ssDNA in the MspA constriction in a similar fashion to a helicase enzyme that steps through double-stranded (dsDNA) in an ONT sequencer, thereby enabling long duration current readings for each sequence tetramer (FIG. 5).

TABLE 1
Chemically modified nucleotides used in the DNA data storage system, along with
their chemical properties.

| Symbol | B1 | B2 | B3 | B4 | B5 | B6 | B7 |
|---|---|---|---|---|---|---|---|
| Name | 2,6- | 5- | 5-hydroxy- | 5- | Deoxyuridine | 5- | 1,2- |
| | Diamino- | Hydroxy- | butynl-2′- | Nitroindole- | | Octadiynyl | Dideoxyribose |
| | purine 2′- | methyl | Deoxyuridine | 2′- | | Deoxyuridine | |
| | deoxyriboside | Deoxycytidine | | Deoxy- | | | |
| | | | | riboside | | | |
| Structurally | dA | dC | dT | dA | dT | dT | — |
| most similar | | | | | | | |
| nucleotide | | | | | | | |
| Pairing mate/ | dT | dG | dA | All | dA | — | — |
| interaction | H | H | H | natural | H | | |
| type (IDT*) | bonds | bonds | bonds | nucleotides | bonds | | |
| | | | | Stacking | | | |
| Pairing mate/ | — | dG | dA | dG | dA | dA, | — |
| interaction | | H | H | Stacking | H | dC H | |
| type | | bonds | bonds | | bonds | bonds | |
| (Simulation**) | | | | | | | |

The symbols and the names of the chemically modified nucleotides are shown in the first and second row, and the molecular structures are depicted in FIG. 1. Structurally similar natural nucleotides are shown in the third row. In general, distinct chemical functional groups and molecular charges play an important role in discriminating monomers using MspA and ONT sequencers. The last two rows show pairing properties of the modified bases: *denotes data from Integrated DNA Technologies while **denotes results from molecular dynamics simulations reported in FIGS. 4, 9-10, and Table 6. Short dashes indicate that pairing is inherently impossible (e.g., B7) or that no stable interactions were identified.

MspA nanopores were used to determine residual currents for homotetramernc sequences of all natural and chemically modified monomers (FIG. 2B3). Our results show that MspA accurately discriminates all four natural (A, G, C, T) and nearly all chemically modified nucleotides (1B1-1B7) at an applied bias of 150 mV. The abasic nucleotide B7 shows the largest residual current, which likely arises due to its small molecular size and reduced ability to interact with the reading head of MspA. The residual current levels are sensitive to the chemical identity of the nucleotides but do not directly correlate with their molecular size (FIG. 2B). For example, current signals from B6 and B2 overlap at 150 mV, but B6 is well separated from B3 despite being structurally similar. The effect of the applied bias on the resolution of nucleotide bases was also studied. At 150 mV, four chemically modified nucleotides (B2, B3, B4, B5) showed well-resolved signals from each other and the natural nucleotides, but the current levels from B6 exhibited some overlap with B2. Upon increasing the applied bias to 180 mV, B6 was readily resolved from B2. In addition, at 180 mV, resolution in the Iᵣₑₛ region exceeding 20% decreased, as may be seen from the residual currents of B4, A, and G which have Gaussian readout distributions which overlap in area by more than 90% (FIG. 2B).

MspA was further used to detect and identify heterotetrameric sequences with compositions 2X+2Y, where X, Y={B2, B3, B4, B5} (FIG. 2C, FIGS. 5-6, Tables 2-4). Our results show that MspA can distinguish all heterotetrameric sequences with the same nucleotide composition when measurements at all three applied biases (150 mV, 180 mV, 200 mV) are performed. Due to the large sequence space explored, the present description includes representative tetrameric combinations of B2 and B3 (FIG. 2C). In most cases, the residual currents of heterotetramers fall between those of two corresponding homotetramers. For example, the tetramer 3223 has an Iᵣₑₛ of 12.3%, whereas those of B2 and B3 are 10.2% and 12.6%, respectively (at 180 mV). However, some combinations of B2 and B3, including 2232, 2322, 2333, 3233, 2323, 2332, and 2233, showed significant decreases in residual currents compared to homotetramers B2 and B3 (FIG. 2C), whereas the residual current of tetramer 3322 is larger than homotetramers of B2 and B2 at either 150 mV or 180 mV. Importantly, all tetrameric sequences were resolved by adjusting the applied bias. At a higher applied bias of 200 mV, tetramers that were unresolved at lower bias were readily resolved, including 2322, 2332, and 2322 (FIG. 2C). Overall, these results are consistent with the observation that the residual current levels of DNA tetramers are not directly correlated with molecular size, similar to the case of natural nucleotides where the blockade current was found to be determined by the competition of steric and base stacking interactions (Manrao, et al. Reading DNA at single-nucleotide resolution with a mutant MspA nanopore and phi29 DNA polymerase. Nat Biotechnol. 2012 April; 30(4):349-53; Bhattacharya et al., Water Mediates Recognition of DNA Sequence via Ionic Current Blockade in a Biological Nanopore. ACS Nano. 2016 Apr. 26; 10(4):4644-51).

The ability of MspA pores to resolve different tetramers containing both natural and chemically modified nucleotides is also described (FIG. 2D). The present disclosure focuses on heterotetramers containing a single chemically modified nucleotide (B2, B3, or B5) added in different positions of the directional sequence ACT. The results clearly show that different positions of the chemically modified nucleotide in the tetramer generates distinct residual currents. For example, the residual current of heterotetrameric sequences of ACT containing four different positions of B2 (2ACT, A2CT, AC2T, and ACT2) are readily resolved at both 150 mV and 180 mV (FIG. 2D). Although the residual current of homotetramer B2 and heterotetramer 2ACT overlap by ˜29% in their Gaussians at 150 mV, they are distinguishable at 180 mV. In addition, nearly all heterotetrameric sequences of ACT containing four different positions of B3 were resolved from the homotetramer B3 at 150 and 180 mV, whereas the residual currents of 3ACT and ACT3 were only distinguishable at 180 mV (FIG. 2D). These results are consistent with prior work reporting that tuning the applied bias is a useful approach to enhance the accuracy of nanopore-based sequencing methods (Noakes et al. Increasing the accuracy of nanopore DNA sequencing using a time-varying cross membrane voltage. Nat Biotechnol. 2019 June; 37(6):651-6). In summary, these results show the ability of MspA nanopores to accurately identify sequences containing chemically modified nucleotides.

In theory, sequence context allows for high-resolution readout of arbitrary combinations and arrangements of natural and modified nucleotides (A, C, G, T, B1-B7). Although specific sets of tetramers might be confused during MspA reading, the method of shift reconciliation allows for such sequences to be fully resolved using the information provided by different shifts of the tetramers within the constriction of the nanopore (FIG. 2E). The concept of shift reconciliation is illustrated with the following example, where a heterogeneous sequence of 23223 is considered. In terms of the corresponding residual current levels, the prefix tetramer 2322 is confusable with 2332 or 2323 at 150 mV. However, by shifting the sliding window one position to the right, the tetramer 3223 is obtained, which is not confusable with any other block. Because the trimer prefix of 3223, 322, only matches the trimer suffix of only one of the tetramers 2322, 2332, 2322 (i.e., the first one), one may unambiguously deduce that 2322 is the correct prefix tetramer.

Moving beyond tetramer detection via MspA, the present disclosure demonstrates that commercially available nanopore-based sequencing technology (ONT GridION) can be used to classify/sequence oligos containing the proposed molecular alphabet. For GridION experiments, the same ssDNA oligos used in MspA experiments were extended at the 3′ terminus with a polyA tail of random length >100 nts, which is used to increase the length of the oligos and guide them inside the pore (FIG. 3A). Raw current signals were retrieved from the GridION platform following a custom RNA sequencing protocol (Methods). Raw current signals were processed using deep learning techniques to discriminate and identify different combinations and orderings of the chemically modified nucleotides. As a first step, regions in the raw current signals corresponding to chemically modified nucleotides were isolated. For this purpose, the specialized software suite Tombo (Timp et al., DNA Base-Calling from a Nanopore Using a Viterbi Algorithm. Biophysical Journal. 2012 May; 102(10): L37-9), designed by ONT for identifying potentially modified nucleotides from nanopore sequencing data was not utilized, as it requires basecalling, alignment and further downstream processing. Accurate basecalling of chemically modified nucleotides is difficult to accomplish which greatly complicates alignment and classification tasks for arbitrary sub-regions of the signal. Moreover, the most recent ONT basecaller, Bonito, based on convolutional neural networks, is trained and specialized to work for natural DNA only (Bonito; A PyTorch Basecaller for Oxford Nanopore Reads. Available from: https://github.com/nanoporetech/bonito). For these reasons, an analysis framework was developed that directly operates on raw current signals of the chemically modified nucleotides.

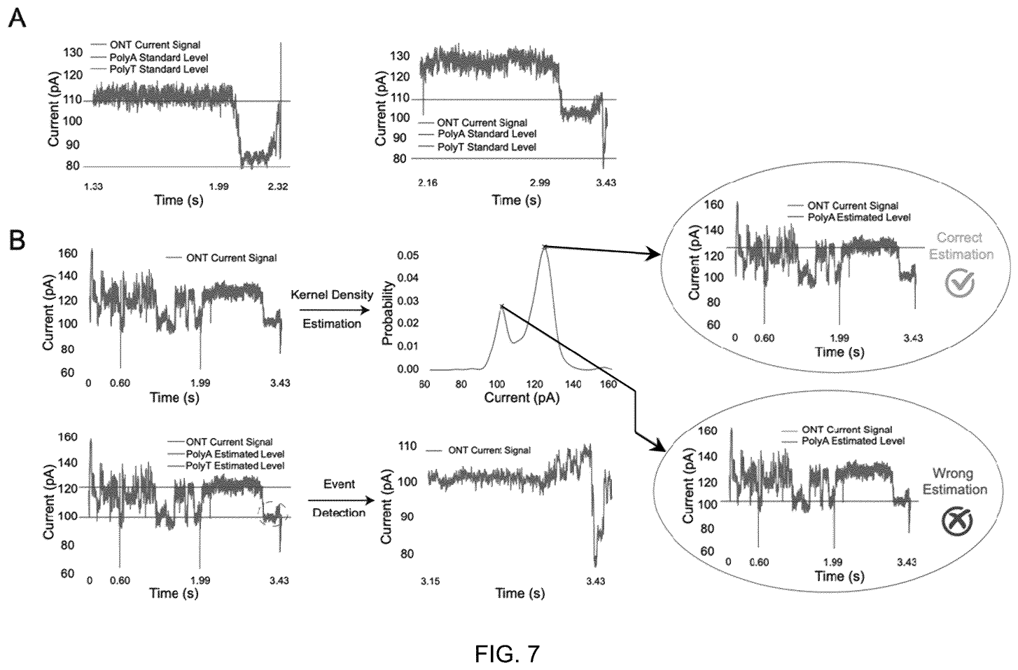

Analysis of raw current signals is challenging because nanopore current signals exhibit extreme variations known as level drifts (FIG. 7). Level drifts arise because each membrane patch (recording channel) inside the device has its own electric circuit, and each pore has unique features. To address this challenge, a two-step identification scheme depicted in FIG. 3B was developed. In the first step, the current level for the polyA region was estimated, and subsequently used for signal calibration. Similar calibration steps are standardly performed for nanopore sequencing of natural DNA, but they rely on adaptor-based calibrations since all analytes use identical adaptors with a well-defined sequence content. For actual level calibration, kernel density estimation of the signal level distribution was utilized, followed by identification of the levels that have the two largest probabilities in the estimated distribution. This approach is justified because polyA regions constitute the longest signal component in our oligo sequences. Moreover, on average, polyT levels are expected to be lower than polyA levels, so readout regions that are trailed by nearly flat regions with a mean level value lower than that for the polyA tails are filtered using a finite state machine. These regions are expected to bear signals from the chemically modified nucleotides. After extracting modification-bearing signals, raw current readouts are subsequently classified. For this task, a 1D residual neural network model was designed (26,27) (FIG. 3C) containing 1D convolution layers (conv) that serve as feature extractors, and one fully connected layer (fc) that serves as a classifier. The model is trained on oligo data corresponding to different combinations and orderings of chemically modified nucleotides, with each option supported by thousands of training samples (Table 5). Elements from each class are uniformly sampled at random in a balanced manner and split into training/validation/test sets with splitting percentages 60%/20%/20%, respectively.

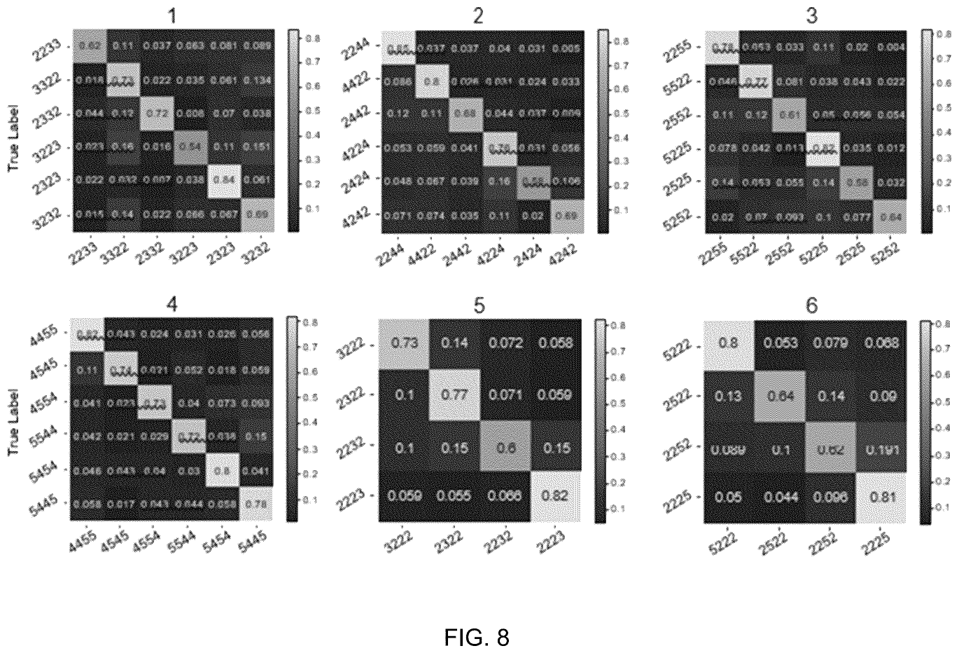

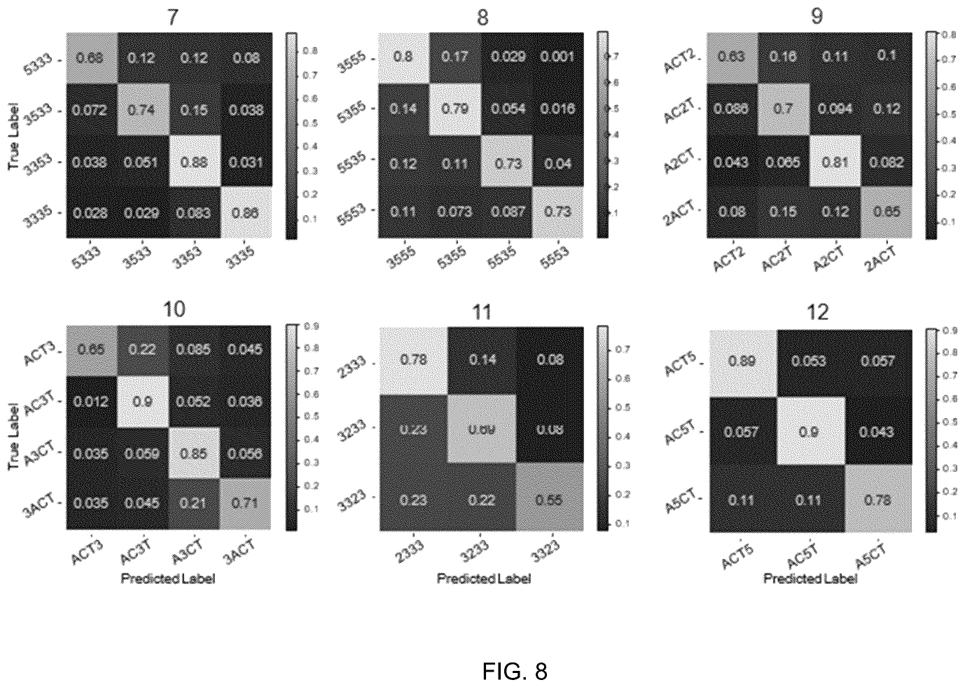

Results from neural network-guided identification tasks pertaining to five independent experimental runs are shown in FIG. 3D. Confusion matrices are used to summarize the prediction accuracies, ranging between 0 and 1 (with 1 corresponding to perfectly accurate identification). Importantly, these results show that most tetramers are identified with high accuracy (i.e., the diagonal elements are significantly larger than the off-diagonal elements). The average classification accuracy for each model is provided in the caption of FIG. 3D, along with the accuracy one would expect from random guessing. For example, an accuracy of 0.85 was observed for heterotetramers (2244, 2244), which is to be interpreted as an 85% success rate in correctly identifying the sequence 2244, or a 15% chance of misinterpreting 2244 as another combination or sequence order (FIG. 3D). Overall, a total of 13 different classification tasks were performed, including one task for all classes (77 in total, from which only 66 were depicted due to small amounts of training data for the remaining 11 classes). Additionally, 12 tasks involving subsets of classes containing chemically modified nucleotides were included as shown in FIG. 1. For brevity, two results for 2X+2Y classes and a summary of all results are shown in FIG. 3D; the full set of results are shown in FIG. 8.

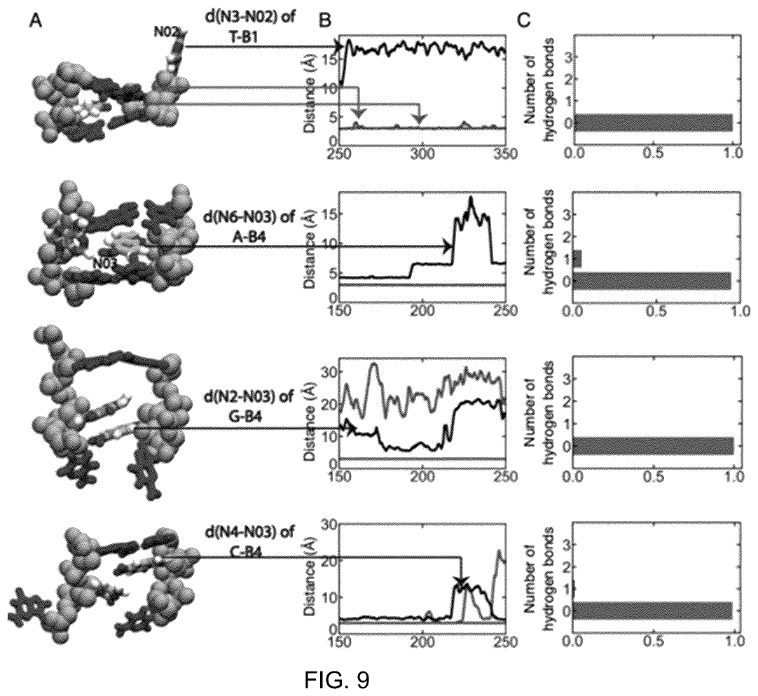

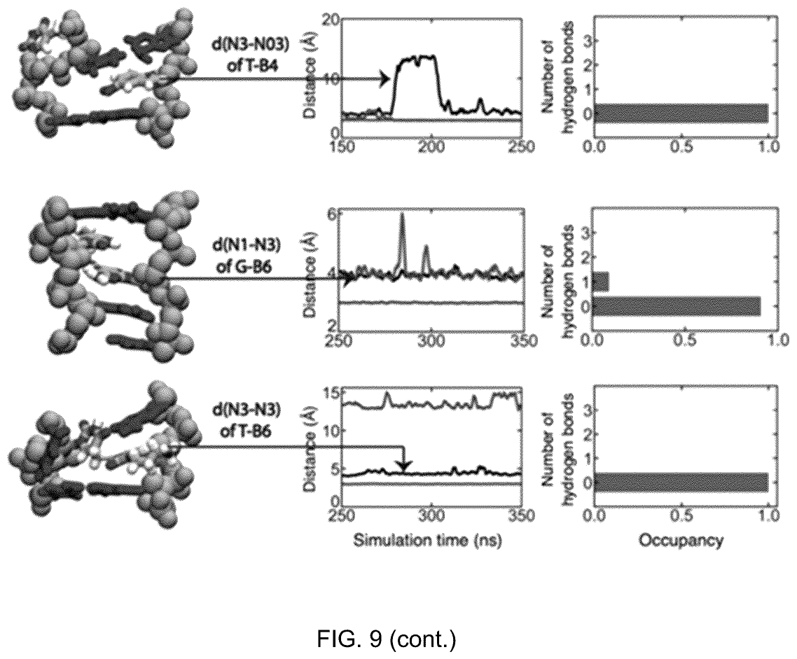

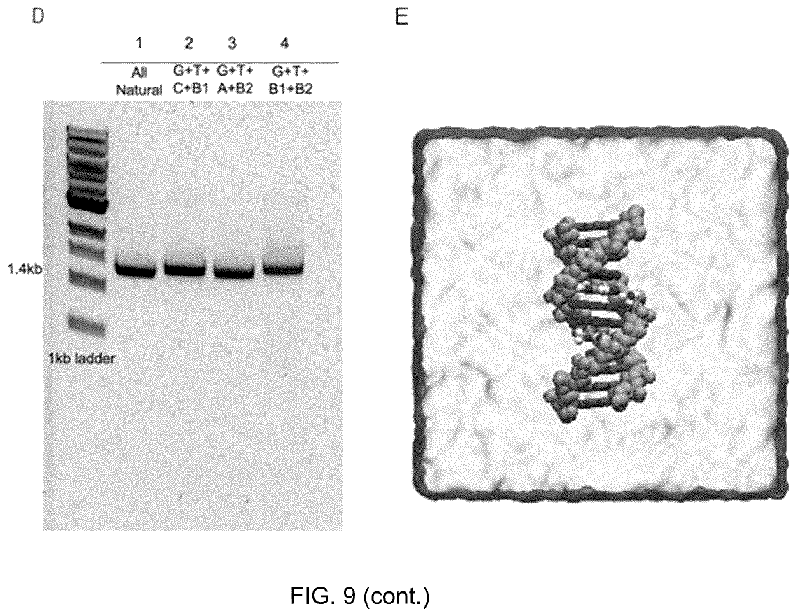

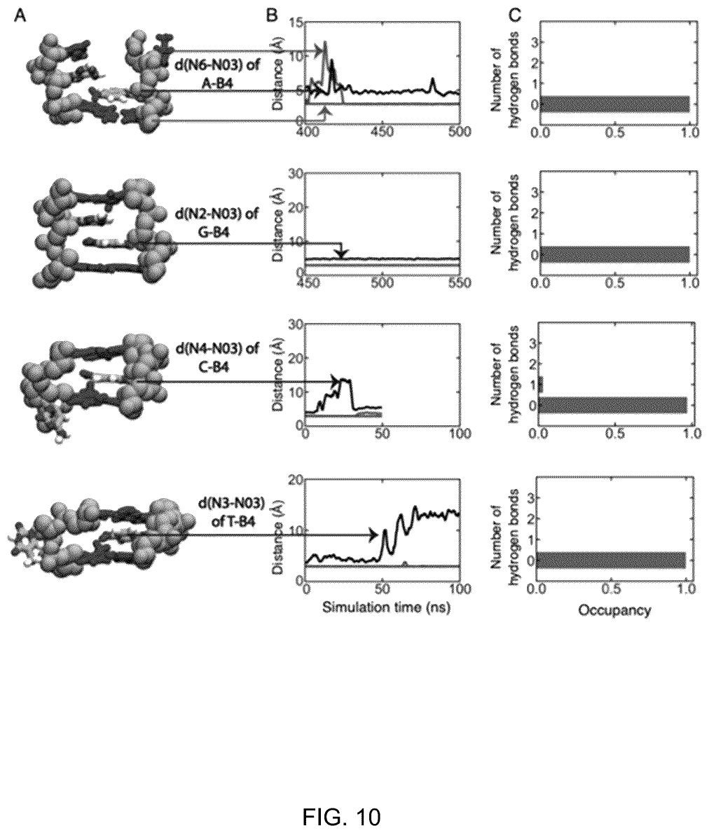

Stable bonding of chemically modified nucleotides within a DNA double helix is important for DNA-based storage because it enables durable preservation of recorded information, as well as random access to the stored data by means of PCR reactions. To better understand the interactions between chemically modified and natural nucleotides, the stability of modified DNA duplexes was investigated by carrying out all-atom molecular dynamics (MD) simulations of the Dickerson dodecamers containing a pair of chemically modified nucleotides (Drew H R, Wing R M, Takano T, Broka C, Tanaka S, Itakura K, et al. Structure of a B-DNA dodecamer: conformation and dynamics. Proceedings of the National Academy of Sciences. 1981 Apr. 1; 78(4):2179-83). Out of many possible variants, the stability of B1-T, B2-G, B3-A, and B5-A base pairs was investigated, as suggested by Integrated DNA Technologies (IDT), as well as the pairing of B4 and B6 with all four types of natural nucleotides. Each modified dodecamer was solvated in electrolyte solution and simulated for approximately 350 ns. Five modified-natural base pairs, (B2-G, B3-A, B5-A, B6-A, and B6-C) were found to form stable hydrogen bond patterns within the duplex forming either two or three hydrogen bonds per base pairs (FIG. 4). The average number of hydrogen bonds was found to be 1.37 for B2-G, 1.01 for B3-A, 1.00 for B5-A, 1.00 for B6A and 0.70 for B6-C, which are results compatible with the numbers computed for the canonical base pairs (0.83 for A-T and 1.23 for C-G) using the same hydrogen bond criteria. In all other modified-natural combinations, local disruptions of the base pairing structure was observed (FIGS. 9-10). In B1-T, B4-A and B4-T pairs, the bases were observed to protrude out from the duplex without disrupting the hydrogen bonding of the surrounding base pairs. The B6-G pair formed a base stacking pattern, forcing the breakage of hydrogen bonds in the adjacent base pairs. Local unraveling of the duplex structure was observed in the systems containing B4-G, B4-C and B6-T base pairs. Based on these results, it is concluded that most of the chemically modified nucleotides introduce minor perturbations to the structure of the duplex except for B4, which does not fit well within the geometry of the classical DNA duplex but is not sufficient to produce a complete unraveling of the DNA duplex. However, it is also observed that an isolated B4-G base pair is able to maintain stable stacking interaction when simulated under conditions that mimic the presence of a longer DNA strand (FIG. 10).

Thus, the enclosed results demonstrate an expanded alphabet for DNA data storage compatible with nanopore sequencing technology. A unique feature of this approach is coupled, iterative selection and testing that involves determining suitability for forming stable duplex structures and nanopore sequencing. Overall, the described system enables the recording of digital data with increased storage density and more bits per synthesis cycle. In particular, the disclosed storage system, when utilizing with 11 unique nucleotides, enables a maximum recording density of log₂11 bits in each cycle, compared to log₂4=2 bits for natural DNA. This strategy also theoretically increased the rate (speed) of the recorder by (log₂11/log₂4)=1.73 fold. Our extensive nanopore experiments provide strong evidence that many more chemically modified nucleotides can be used for molecular storage because many ionic current levels remain available, i.e., the ionic current spectrum is sparsely populated. In addition, our system allows for high-fidelity readouts and PCR-based random-access features for encodings restricted to duplex formation competent monomers. Although not all pairings of chemical modifications may be suitable for amplification using natural enzymes, and some duplex formations may be unstable, the proposed system provides the first example of a coupled coding alphabet and channel selection and optimization paradigm. In conclusion, this work demonstrates fundamentally new directions in molecular storage that hold the potential to advance the field of DNA-based data storage.

Materials and Methods

Oligo design and synthesis. All oligos tested are of fixed length 40 nt and synthesized by Integrated DNA Technologies (IDT). For MspA experiments, the content of the oligos was chosen to include two polyT sequences at locations 1-12 and 17-40, and a chemically modified tetramer at positions 13-16. All oligos were biotinylated at the 5′ end.

PCR Amplification. DNA amplification was performed via PCR using Q5 DNA polymerase, 5×Q5 buffer and pUC19 plasmid as template (New England Biolabs) in 50 μl. The 1.4 kb sequence is:

(SEQ ID NO: 01)
5′CGTTTTACAACGTCGTGACTGGGAAAACCCTGGCGTTACCCAACTTA
ATCGCCTTGCAGCACATCCCCCTTTCGCCAGCTGGCGTAATAGCGAAGA
GGCCCGCACCGATCGCCCTTCCCAACAGTTGCGCAGCCTGAATGGCGAA
TGGCGCCTGATGCGGTATTTTCTCCTTACGCATCTGTGCGGTATTTCAC
ACCGCATATGGTGCACTCTCAGTACAATCTGCTCTGATGCCGCATAGTT
AAGCCAGCCCCGACACCCGCCAACACCCGCTGACGCGCCCTGACGGGCT
TGTCTGCTCCCGGCATCCGCTTACAGACAAGCTGTGACCGTCTCCGGGA
GCTGCATGTGTCAGAGGTTTTCACCGTCATCACCGAAACGCGCGAGACG
AAAGGGCCTCGTGATACGCCTATTTTTATAGGTTAATGTCATGATAATA
ATGGTTTCTTAGACGTCAGGTGGCACTTTTCGGGGAAATGTGCGCGGAA
CCCCTATTTGTTTATTTTTCTAAATACATTCAAATATGTATCCGCTCAT
GAGACAATAACCCTGATAAATGCTTCAATAATATTGAAAAAGGAAGAGT
ATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGA
TGCTGAAGATCAGTTGGGTGCACGAGTGGGTTACATCGAACTGGATCTC
AACAGCGGTAAGATCCTTGAGAGTTTTCGCCCCGAAGAACGTTTTCCAA
TGATGAGCACTTTTAAAGTTCTGCTATGTGGCGCGGTATTATCCCGTAT
TGACGCCGGGCAAGAGCAACTCGGTCGCCGCATACACTATTCTCAGAAT
GACTTGGTTGAGTACTCACCAGTCACAGAAAAGCATCTTACGGATGGCA
TGACAGTAAGAGAATTATGCAGTGCTGCCATAACCATGAGTGATAACAC
TGCGGCCAACTTACTTCTGACAACGATCGGAGGACCGAAGGAGCTAACC
GCTTTTTTGCACAACATGGGGGATCATGTAACTCGCCTTGATCGTTGGG
AACCGGAGCTGAATGAAGCCATACCAAACGACGAGCGTGACACCACGAT
GCCTGTAGCAATGGCAACAACGTTGCGCAAACTATTAACTGGCGAACTA
CTTACTCTAGCTTCCCGGCAACAATTAATAGACTGGATGGAGGCGGATA
AAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCA
GCACTGGGGCCAGATGGTAAGCCCTCCCGTATCGTAGTTATCTACACGA
CGGGGAGTCAGGCAACTATGGATGAACGAAATAGACAGATCGCTGAGAT
AGGTGCCTCACTGATTAAGCATTGGTA3′.

All primers were purchased from Integrated DNA Technologies (IDT). Both B1 and B2 were purchased from TriLink Biotechnologies in the form of triphosphates (https://www.trilinkbiotech.com/2-amino-2-deoxyadenosine-5-triphosphate-n-2003.html and https://www.trilinkbiotech.com/5-hydroxymethyl-2-deoxycytidine-5-triphosphate.html). All natural and chemically modified nucleotides were added in equimolar ratios in all PCR reactions.

MD Simulations. The molecular mechanics models of modified nucleotides B1, B3, B4, B5 and B6, including their topology and force field parameter files, were generated using the CHARMM General Force Field (CGenFF) (Vanommeslaeghe, et al. CHARMM general force field: A force field for drug-like molecules compatible with the CHARMM all-atom additive biological force fields. J Comput Chem. 2009). The charge of the atom connecting to the sugar was adjusted so that the total charge of the base is zero, which is the case for all the natural nucleotides in CHARMM36. The parameters for B2 were adopted from a previous study (Frauer, et al. Recognition of 5-Hydroxymethylcytosine by the Uhrfl SRA Domain. Xu S, editor. PLoS ONE. 2011 Jun. 22; 6 (6): e21306). Eight systems each containing a modified Dickerson dodecamers (CGCGAATTCGCG) (SEQ ID NO:02) (Drew H R, et al. Structure of a B-DNA dodecamer: conformation and dynamics. Proceedings of the National Academy of Sciences. 1981 Apr. 1; 78(4):2179-83.) were created starting from a B-DNA conformation to contain two different pairs of modified and natural bases while all other bases remained as in the original sequence. Each DNA duplex was immersed in a 75 Å×75 Å×75 Avolume of 1M KCl solution. After 2000 steps of energy minimization, the systems were equilibrated with the DNA backbone phosphate atoms restrained (ks=1 kcal/mol/Å²) for the first 10 ns. Each system contains approximately 39,000 atoms. Additional restrains were applied to enforce the expected hydrogen bonds between the modified and natural nucleotides for the first 20 ns. The systems were simulated for 350 ns in the absence of any restrains in the constant number of particles, pressure (1 atm) and temperature (295 K) ensemble using NAMD2 (Phillips J C, Hardy D J, Maia J D C, Stone J E, Ribeiro J V, Bernardi R C, et al. Scalable molecular dynamics on CPU and GPU architectures with NAMD. J Chem Phys. 2020 Jul. 28; 153(4):044130). If prominent structural disruptions had developed in both base pairs surrounding the modified nucleotide base pair, the simulation was terminated. Specifically, the simulation of the systems containing the B4 nucleotide lasted only 250 ns. Simulations of all the systems were performed using periodic boundary conditions. The simulations employed the particle mesh Ewald (PME) algorithm (Darden T, York D, Pedersen L. Particle mesh Ewald: An N·log(N) method for Ewald sums in large systems. The Journal of Chemical Physics, 1993, 98(12):10089-92) to calculate long-range electrostatic interaction over a 1 Å-spaced grid. RATTLE (Andersen H C. Rattle: A “velocity” version of the shake algorithm for molecular dynamics calculations. Journal of Computational Physics. 1983 October; 52(1):24-34) and SETTLE (Miyamoto S, Kollman P A. Settle: An analytical version of the SHAKE and RATTLE algorithm for rigid water models. J Comput Chem. 1992 October; 13(8):952-62) algorithms were adopted to constrain all covalent bonds involving hydrogen atoms, allowing 2-fs time step integration used in the simulations. van der Waals interactions were calculated using a smooth 10-12 Å cutoff. The NPT ensembles used the Nose-Hoover Langevin piston pressure control (Martyna G J, Tobias D J, Klein M L. Constant pressure molecular dynamics algorithms. The Journal of Chemical Physics. 1994 September; 101(5):4177-89), which maintained a constant pressure by adjusting system's dimension. Simultaneously, Langevin thermostat was adopted for temperature control, with damping coefficient of 0.5 ps applied to all heavy atoms in the systems. CHARMM36 (Hart K, et al., Optimization of the CHARMM Additive Force Field for DNA: Improved Treatment of the BI/BII Conformational Equilibrium. J Chem Theory Comput. 2012 Jan. 10; 8(1):348-62), output of CGenFF, TIP3P water model as long as custom NBFIX corrections to nonbonded interactions were employed as the parameter set of the simulation. The hydrogen bonds occupancy, the distances between hydrogen bond donors and acceptors as well as the short/long axis lengths of bases are calculated from the well equilibrated last 100 ns fragment of the trajectory using VMD (Humphrey W, Dalke A, Schulten K. VMD: Visual molecular dynamics. Journal of Molecular Graphics. 1996 February; 14(1):33-8). The hydrogen bonds were defined to have the donor-accepter interaction distance of less than 3A and the cutoff angle of 20°. Given the largely planar shape of the bases, their short/long were determined by first computing the three principal axes of the bases and then choosing the largest two values. Simulations/analysis of the B4 pairing with natural bases in longer DNA strands were conducted using the same methodology, but with only one modified base contained in the dodecamer. Besides, extra bonds were applied to the donor (N1) and accepter (N3) atoms on the terminal pairs to prevent the ends from fraying in these simulations to adapt the situation of long DNA strands. These simulations ran 550 ns except if unstable configurations were observed.

MspA nanopores and purification of M2-NNN MspA. All chemicals were purchased from Fisher Scientific unless stated otherwise. Streptavidin was ordered from EMD Millipore (Burlington, MA) (Catalog #189730). Phenylmethylsulfonyl fluoride (PMSF) was ordered from GoldBio (St. Louis, MO) (Catalog #P-470). DNA of M2-NNN MspA construct was a gift from Dr. Giovanni Maglia (University of Groningen, Netherlands). The pT7-M2-NNN-MspA was transformed into BL21 (DE3) pLyss cells and grown in LB medium at 37° C. until the OD600 reached 0.5-0.6. The cells were then induced with 0.5 mM isopropyl P-D-1-thiogalactopyranoside (IPTG) and continued to grow at 16° C. for 16 hours. Cells were harvested and centrifuged at 19,000×g for 30 min at 4° C. Cells were resuspended in the lysis buffer containing 100 mM Na₂HPO₄/NaH₂PO₄, 1 mM ethylenediaminetetraacetic acid (EDTA), 150 mM NaCl, 1 mM phenylmethylsulfonyl fluoride (PMSF) pH 6.5, before heating at 60° C. for 10 minutes. The cells were sonicated by using VWR Scientific Branson 450 sonicator (duty cycle of 20% and output control of 2) for 8 minutes. The lysate was centrifuged at 19,000×g for 30 min and the supernatant was discarded. The pellet was resuspended in the solubilization buffer containing 100 mM Na₂HPO₄/NaH₂PO₄, 1 mM EDTA, 150 mM NaCl, 0.5% (v/v) Genapol X −80, pH 6.5. After completely resuspending the pellet, it was centrifuged at 19,000×g for 30 min. The supernatant, containing solubilized membrane extract, was collected for Ni-NTA purification. MspA was further purified using a 5 mL HisPur™ Ni-NTA resin (GE Healthcare) and eluted in a buffer of 0.5 M NaCl, 20 mM HEPES, 0.5% (v/v) Genapol X −80, pH 8.0 by applying an imidazole gradient. MspA oligomers were further purified by SDS-PAGE gel extraction. The purified MspA protein was run in 7.5% SDS-PAGE gel. The band of MspA oligomer was cut from the gel and extracted in the extraction buffer containing 50 mM Tris-HCl, 150 mM NaCl, 0.5% Genapol X −80, pH 7.5. The protein was extracted at room temperature (23° C.) for 6 hours before centrifuged at 9,000×g for 30 min to collect the protein solution. The purified MspA oligomer was fast frozen and stored at −80° C. for further use.

Single-channel recording using MspA. The experiments were performed in a device containing two chambers separated by a 25 μm thick polytetrafluoroethylene film (Goodfellow) with an aperture of approximately 100 μm diameter located at the center. A hexadecane/pentane (10% v/v) solution was first added to cover both sides of the aperture. After the pentane evaporated, each chamber was then filled with buffer containing 1 M KCl 10 mM HEPES pH 8.0. 1, 2-diphytanoyl-sn-glycero-3-phosphocholine (DPhPC) dissolved in pentane (10 mg/mL) was dropped on the surface of the buffer in both chambers. After the pentane evaporated, the lipid bilayer was formed by pipetting the solution in both chambers below the aperture several times. An Ag/AgCl electrode was immersed in each chamber with the cis side grounded. M2-NNN MspA proteins (around 1 nM, final concentration) were also added to the cis chamber. To promote MspA insertion, a≥+200 mV voltage was applied. After a single MspA was inserted into the planar lipid bilayer, the applied voltage was decreased to 150 mV (or 180 mV) for recording. The current was amplified with an Axopatch 200B integrating patch-clamp amplifier (Axon Instruments, Foster City, CA). Signals were filtered with a Bessel filter at 2 kHz and then acquired by a computer (sampling at 100 s) after digitization with a Digidata 1440A/D board (Axon Instruments).

DNA immobilized in MspA. After recording a single MspA pore for 5-10 minutes at positive voltages to check its stability, 5′-biotinylated DNA sample (final concentration of 0.25 μM) was added to the cis chamber. Streptavidin (0.1 μM), added to solutions in the cis chamber, can bind to biotin to prevent the full translocation of the DNA strand through the nanopore. To collect the signal generated from each DNA samples, a sweep protocol was applied. The amplifier applied either 150 mV or 180 mV for 10 s then applied −150 mV to force the DNA out of the pore back into the cis compartment. The voltage was then returned to the original value and the sweep protocol repeated for at least 40 times at each voltage.

ONT sequencing protocol. NEB terminal transferase was used for A-tailing the 3′ end of the 40-mer control oligos. The reaction mixture was made by 5 ul 10×TdT buffer, 5 ul 2.5 mM CoCl2, 5 pmole DNA, 0.5 ul 10 mM dATP, 0.5 ul terminal transferase, and 38 ul H₂O. The reaction was Incubated at 37° C. for 30 mins, followed by inactivation at 70° C. for 10 mins. The DNA was then purified using the Zymo DNA clean up kit (ssDNA Buffer:sample=7:1) and eluted in 10 ul warm H₂O. The Oxford Nanopore SQK-RNA002 kit was used for library preparation.

The RT adaptor was ligated for 10 min at room temperature, then mixed with reverse transcription master mix. 2 uL of Superscript IV were added and the mixture was Incubated at 50 C for 50 mins, followed by 70° C. for 0 mins and cooled down to 4° C. Bead clean-up was performed using 40 ul samples with 72 ul RNAClean XIP beads, rotated for 5 mins, washed by 70% EtOH and eluted by 20 ul H₂O. The RMVX adaptor was ligated in 10 mins at room temperature, then 40 ul RNA Clean XIP beads clean-up was used, and the product was washed with 150 ul of the wash buffer twice. It was then eluted in 21 ul of the elution buffer. The reaction was loaded onto an R9.4.1 flowcell and sequenced on a GridION X5 (Oxford Nanopore) for 24 hs.

TABLE 2
The mean residual currents (Iᵣₑₛ (%)) and the full-width half-height
(FWHM) values for each oligonucleotide were determined by Gaussian
fitting of the residual current histogram from experiments with
different combination of natural and modified nucleotides at positions
13-16 from the streptavidin anchor at 150 mV.

| Combination | X | Y | Sample | Iᵣₑₛ (%) | FWHM |
|---|---|---|---|---|---|
| ACT + X | 2 | | 2ACT | 8.68 | 0.60 |
| | | | A2CT | 10.65 | 0.22 |
| | | | AC2T | 10.14 | 0.67 |
| | | | ACT2 | 9.01 | 0.32 |
| | 3 | | 3ACT | 9.60 | 0.36 |
| | | | A3CT | 10.27 | 0.70 |
| | | | AC3T | 8.69 | 0.41 |
| | | | ACT3 | 9.52 | 0.48 |
| | 5 | | 5ACT | 9.68 | 0.43 |
| | | | A5CT | 13.62 | 0.50 |
| | | | AC5T | 9.90 | 0.38 |
| | | | ACT5 | 9.59 | 0.28 |
| 4X | 1 | | B1 | 19.66 | 0.39 |
| | 2 | | B2 | 8.43 | 0.13 |
| | 3 | | B3 | 10.75 | 0.18 |
| | 4 | | B4 | 22.74 | 0.51 |
| | 5 | | B5 | 15.32 | 0.33 |
| | 6 | | B6 | 8.49 | 0.29 |
| | 7 | | B7 | 31.30 | 0.12 |
| | A | | A | 19.94 | 0.29 |
| | C | | C | 9.84 | 0.13 |
| | G | | G | 20.82 | 0.50 |
| | T | | T | 14.10 | 0.14 |
| 3X + Y | 2 | 3 | 2223 | 9.13 | 0.34 |
| | | | 2232 | 7.36 | 0.38 |
| | | | 2322 | 8.34 | 0.37 |
| | | | 3222 | 9.45 | 0.29 |
| | | 5 | 2225 | 9.45 | 0.55 |
| | | | 2252 | 9.75 | 0.14 |
| | | | 2522 | 9.83 | 0.27 |
| | | | 5222 | 9.83 | 0.48 |
| | 3 | 2 | 2333 | 7.91 | 0.19 |
| | | | 3233 | 7.48 | 0.30 |
| | | | 3323 | 9.44 | 0.29 |
| | | | 3332 | 10.45 | 0.42 |
| | | 5 | 3335 | 11.45 | 0.18 |
| | | | 3353 | 12.37 | 0.27 |
| | | | 3533 | 12.30 | 0.19 |
| | | | 5333 | 12.61 | 0.20 |
| | 5 | 2 | 2555 | 9.39 | 0.37 |
| | | | 5255 | 9.46 | 0.60 |
| | | | 5525 | 11.80 | 0.25 |
| | | | 5552 | 14.35 | 0.55 |
| | | 3 | 3555 | 15.69 | 0.25 |
| | | | 5355 | 13.96 | 0.28 |
| | | | 5535 | 13.43 | 0.27 |
| | | | 5553 | 14.29 | 0.34 |
| 2X + 2Y | 2 | 3 | 2323 | 8.53 | 0.17 |
| | 2 | | 2332 | 8.07 | 0.14 |
| | | | 3223 | 10.02 | 0.16 |
| | | | 3232 | 8.18 | 0.16 |
| | | | 3322 | 11.34 | 0.17 |
| | | | 2233 | 7.59 | 0.14 |
| | | 4 | 2424 | 12.79 | 0.20 |
| | | | 2442 | 13.01 | 0.59 |
| | | | 4224 | 12.39 | 0.12 |
| | | | 4242 | 12.62 | 0.19 |
| | | | 4422 | 12.99 | 0.21 |
| | | | 2244 | 10.78 | 0.18 |
| | | 5 | 2525 | 9.23 | 0.13 |
| | | | 2552 | 10.45 | 0.17 |
| | | | 5225 | 10.03 | 0.09 |
| | | | 5252 | 9.95 | 0.14 |
| | | | 5522 | 10.96 | 0.20 |
| | | | 2255 | 9.89 | 0.13 |
| | 4 | 5 | 4545 | 23.07 | 0.34 |
| | | | 5454 | 20.16 | 0.43 |
| | | | 4554 | 19.55 | 0.20 |
| | | | 5445 | 19.38 | 0.32 |
| | | | 5544 | 17.63 | 0.24 |
| | | | 4455 | 22.01 | 0.33 |
| | 1 | 2 | 1122 | 11.18 | 0.27 |
| | | 3 | 1133 | 16.16 | 0.22 |
| | | 4 | 1144 | 18.09 | 0.30 |
| | | 5 | 1155 | 17.57 | 0.21 |
| | 3 | 4 | 3344 | 19.07 | 0.85 |
| | | 5 | 3355 | 13.32 | 0.19 |

TABLE 3
The mean residual currents (Iᵣₑₛ (%)) and the full-width half-height
(FWHM) values for each oligonucleotide, determined by performing
Gaussian fitting of the residual current histogram from experiments
involving different combination of natural and modified nucleotides
at positions 13-16 from the streptavidin anchor at 180 mV.

| Combination | X | Y | Sample | Iᵣₑₛ (%) | FWHM |
|---|---|---|---|---|---|
| ACT + X | 2 | | 2ACT | 11.06 | 0.49 |
| | | | A2CT | 12.93 | 0.21 |
| | | | AC2T | 12.02 | 0.59 |
| | | | ACT2 | 10.53 | 0.28 |
| | 3 | | 3ACT | 12.38 | 0.38 |
| | | | A3CT | 14.27 | 0.61 |
| | | | AC3T | 10.74 | 0.40 |
| | | | ACT3 | 11.38 | 0.42 |
| | 5 | | 5ACT | 12.07 | 0.43 |
| | | | A5CT | 18.44 | 0.44 |
| | | | AC5T | 12.17 | 0.34 |
| | | | ACT5 | 11.58 | 0.25 |
| 4X | 1 | | B1 | 22.52 | 0.25 |
| | 2 | | B2 | 10.15 | 0.14 |
| | 3 | | B3 | 12.62 | 0.18 |
| | 4 | | B4 | 23.25 | 0.54 |
| | 5 | | B5 | 17.51 | 0.26 |
| | 6 | | B6 | 9.90 | 0.27 |
| | 7 | | B7 | 34.13 | 0.19 |
| | A | | A | 23.07 | 0.30 |
| | C | | C | 11.93 | 0.16 |
| | G | | G | 23.57 | 0.49 |
| | T | | T | 16.40 | 0.20 |
| 3X + Y | 2 | 3 | 2223 | 11.07 | 0.22 |
| | | | 2232 | 8.97 | 0.33 |
| | | | 2322 | 9.64 | 0.26 |
| | | | 3222 | 11.54 | 0.26 |
| | | 5 | 2225 | 11.16 | 0.49 |
| | | | 2252 | 11.36 | 0.13 |
| | | | 2522 | 11.19 | 0.22 |
| | | | 5222 | 11.48 | 0.35 |
| | 3 | 2 | 2333 | 9.25 | 0.16 |
| | | | 3233 | 9.69 | 0.26 |
| | | | 3323 | 12.34 | 0.24 |
| | | | 3332 | 12.64 | 0.39 |
| | | 5 | 3335 | 13.36 | 0.16 |
| | | | 3353 | 14.38 | 0.19 |
| | | | 3533 | 14.45 | 0.22 |
| | | | 5333 | 14.54 | 0.19 |
| | 5 | 2 | 2555 | 11.78 | 0.33 |
| | | | 5255 | 11.48 | 0.45 |
| | | | 5525 | 15.31 | 0.22 |
| | | | 5552 | 17.62 | 0.42 |
| | | 3 | 3555 | 17.59 | 0.35 |
| | | | 5355 | 16.02 | 0.19 |
| | | | 5535 | 15.82 | 0.21 |
| | | | 5553 | 17.14 | 0.28 |
| 2X + 2Y | 2 | 3 | 2323 | 9.65 | 0.15 |
| | 2 | 3 | 2332 | 9.60 | 0.17 |
| | | | 3223 | 12.15 | 0.17 |
| | | | 3232 | 10.05 | 0.17 |
| | | | 3322 | 13.66 | 0.18 |
| | | | 2233 | 9.86 | 0.15 |
| | | 4 | 2424 | 14.14 | 0.22 |
| | | | 2442 | 15.94 | 0.36 |
| | | | 4224 | 14.57 | 0.17 |
| | | | 4242 | 15.22 | 0.18 |
| | | | 4422 | 15.80 | 0.35 |
| | | | 2244 | 12.58 | 0.15 |
| | | 5 | 2525 | 10.57 | 0.09 |
| | | | 2552 | 11.85 | 0.19 |
| | | | 5225 | 12.15 | 0.10 |
| | | | 5252 | 11.55 | 0.09 |
| | | | 5522 | 14.48 | 0.19 |
| | | | 2255 | 11.19 | 0.16 |
| | 4 | 5 | 4545 | 25.65 | 0.41 |
| | | | 5454 | 20.99 | 0.38 |
| | | | 4554 | 22.27 | 0.28 |
| | | | 5445 | 20.74 | 0.45 |
| | | 5 | 5544 | 19.56 | 0.26 |
| | | | 4455 | 23.70 | 0.41 |
| | 1 | 2 | 1122 | 14.05 | 0.24 |
| | | 3 | 1133 | 18.93 | 0.21 |
| | | 4 | 1144 | 21.09 | 0.23 |
| | | 5 | 1155 | 20.50 | 0.25 |
| | 3 | 4 | 3344 | 20.18 | 0.87 |
| | | 5 | 3355 | 14.83 | 0.20 |

TABLE 4
The mean residual currents (Iᵣₑₛ (%)) and the full width half height
values (FWHM) for each oligonucleotide were determined by performing
Gaussian fits to the residual current histogram from experiments
with different combination of natural and modified nucleotides at
position x = 13 − 16 from the streptavidin anchor at 200 mV.

| Combination | X | Y | Sample | Iᵣₑₛ (%) | FWHM |
|---|---|---|---|---|---|
| ACT + X | 2 | | 2ACT | 12.08 | 0.33 |
| | | | ACT2 | 11.57 | 0.44 |
| | 5 | | 5ACT | 15.12 | 0.40 |
| | | | AC5T | 13.78 | 0.51 |
| | | | ACT5 | 12.26 | 0.30 |
| 4X | 2 | | B2 | 12.08 | 0.11 |
| | 2 | 3 | 2322 | 9.93 | 0.11 |
| | | 5 | 2225 | 11.60 | 0.61 |
| | | | 2252 | 12.59 | 0.09 |
| | | | 2522 | 12.21 | 0.16 |
| | | | 5222 | 13.25 | 0.10 |
| 3X + Y | 3 | 2 | 2333 | 10.00 | 0.16 |
| | | 5 | 3353 | 15.47 | 0.41 |
| | | | 3533 | 16.22 | 0.34 |
| | | | 5333 | 12.85 | 0.34 |
| | 5 | 2 | 2555 | 12.39 | 0.49 |
| | | | 5255 | 13.63 | 0.75 |
| 2X + 2Y | 2 | 3 | 2323 | 10.57 | 0.16 |
| | | | 2332 | 10.35 | 0.17 |
| | | | 3232 | 10.86 | 0.26 |
| | | 4 | 2442 | 17.19 | 0.25 |
| | | | 4422 | 17.98 | 0.55 |

Two-Step Event Identification Scheme for ONT Readouts with NN Processing

The main challenges faced when analyzing nanopore current signals are illustrated in FIG. 7. The figure shows the extreme variations in the current levels, which can either stay close to the mean (as illustrated on the example CCCC) or deviate more than 15% from the mean (as illustrated on the example 2233). Therefore, to automatically extract the regions from the ONT current readouts that correspond to modified nucleotides without resorting to basecalling, a two-step identification scheme was developed as depicted in FIG. 7. The first step is to estimate the current level for the polyA region, which is subsequently used for calibration purposes. A kernel density estimation of the signal level distribution was performed, followed by identification of the levels that have the two largest probabilities in the estimated distribution. This approach is justified by the observation that in our oligo structure, the polyA regions constitute the longest signal component. As polyT current levels are expected to be lower than polyA levels, readout regions that are trailed by nearly flat regions with a mean level value lower than that observed for the polyA tails were filtered out using a finite state machine (Stoddart D, Heron A J, Mikhailova E, Maglia G, Bayley H. Single-nucleotide discrimination in immobilized DNA oligonucleotides with a biological nanopore. Proceedings of the National Academy of Sciences of the United States of America. 2009). These regions are expected to bear the signal from the chemically modified nucleotides.

Summary of Results from Model-Based Classification Procedure

ResNet models were trained on 12 permutation classes in which the composition is fixed, but the orderings of the modified nucleotides are different. What is referred to as a “superclass” combines different choices and orderings of the modified nucleotides (the superclass contains 66 out of 77 tetramers, as for 11 tetramers an insufficient number of training samples was available). The number of valid sequenced reads (i.e., reads containing modified nucleotides) for each class is shown in Table 5. To perform unbiased training, the sizes of the classes was balanced by setting a lower bound for subsampling of reads in different classes. An upper bound was also set on the number of training samples used for each class, in order to prohibit one/several classes to dominant the training set. For finer classification involving permutations of monomers within a class, the lower bound was set to 1000, and the upper bound to 5000. For the classification task on all 66 classes, the lower bound was set to 2000, and the upper bound to 3500. These choices are necessitated by two conflicting requirements: To balance out the class sizes and retain a training set as large as possible. The classification results are shown in FIG. 8. From the confusion matrices, almost all combinations were observed to be easily distinguished from each other with very high accuracies (i.e., the diagonal values are significantly larger than the off-diagonal values). However, there are some tetramer instances that are hard to classify, such as 3223 (when compared to a tetramer in {2233, 3322, 2332, 3223, 2323, 3232}). The average classification accuracies for each model trained are listed in the caption of FIG. 8.

TABLE 5
The number of valid reads for each tetramer class (77 classes in total), arranged
in ascending order.

| | Number | | Number | | Number | | Number | | Number |
|---|---|---|---|---|---|---|---|---|---|
| Class | of valid | Class | of valid | Class | of valid | Class | of valid | Class | of valid |
| Name | reads | Name | reads | Name | reads | Name | reads | Name | reads |
| 3332 | 39 | 5255 | 74 | 2555 | 204 | 5ACT | 315 | 7777 | 712 |
| 5525 | 750 | TTTT | 1390 | ACT3 | 1717 | 3323 | 1808 | 3555 | 1885 |
| 5552 | 1944 | A5CT | 2133 | 5535 | 2315 | 3233 | 2344 | 5333 | 2430 |
| 5553 | 2460 | 4444 | 2553 | GGGG | 2607 | 6666 | 2632 | 2424 | 2706 |
| 1144 | 2723 | 4422 | 2740 | 1133 | 3134 | 3353 | 3167 | 4242 | 3310 |
| 4224 | 3377 | 3223 | 3732 | ACT2 | 3837 | 3322 | 3865 | 2442 | 3967 |
| 2255 | 4039 | 4545 | 4072 | 4455 | 4500 | 3333 | 4506 | 5555 | 4630 |
| 5225 | 4657 | 4554 | 4827 | 2ACT | 4827 | 1122 | 4844 | 5355 | 4925 |
| A2CT | 5197 | CCCC | 5198 | 5522 | 5236 | 3232 | 5324 | 3ACT | 5403 |
| 5544 | 5485 | AC2T | 5505 | 2333 | 5612 | 5222 | 5905 | 2222 | 5958 |
| 5454 | 6090 | 5445 | 6163 | 3222 | 6395 | 2244 | 6484 | 2252 | 6509 |
| 3533 | 6526 | AC5T | 6532 | 3355 | 6556 | 2522 | 6799 | 2233 | 7047 |
| 2525 | 7403 | A3CT | 7448 | 2225 | 7563 | 1155 | 7591 | 2223 | 7700 |
| 3344 | 7716 | AAAA | 7927 | 3335 | 7952 | 2552 | 9525 | 2232 | 9955 |
| ACT5 | 11768 | 1111 | 13502 | 2322 | 13915 | 2323 | 15927 | 5252 | 16104 |
| 2332 | 17890 | AC3T | 22040 | | | | | | |

While particular aspects and embodiments are disclosed herein, other aspects and embodiments will be apparent to those skilled in the art in view of the foregoing teaching. The various aspects and embodiments disclosed herein are for illustration purposes only and are not intended to be limiting, with the true scope and spirit being indicated by the following claims.

We claim:

1. A DNA data storage system comprising a covalently linked sequence of nucleotides, wherein the sequence of nucleotides comprises a modification region, wherein the nucleotides comprise synthetic nucleotides, wherein the synthetic nucleotides are each independently of the formula: 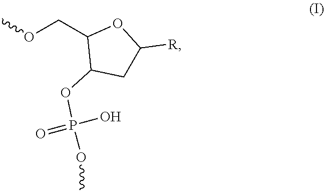
wherein R is H, or is a heterocycle.

2. The DNA data storage system of claim 1, wherein, when R is not H, R is capable of making at least 1 hydrogen bond to a natural nucleotide.

3. The DNA data storage system of claim 1, wherein R is H or a nitrogen-containing heterocycle, wherein the heterocycle is monocyclic or fused bicyclic.

4. The DNA data storage system any of claim 1, wherein R is H,

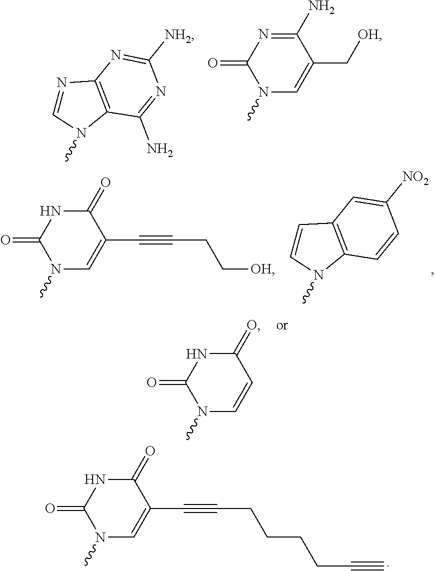

5. The DNA data storage system of claim 1, wherein the sequence of nucleotides further comprises a 5′-bound biotin.

6. The DNA data storage system of claim 5, further comprising streptavidin bound to the biotin.

7. The DNA data storage system of claim 1, wherein the covalently linked sequence of nucleotides comprises a calibration region.

8. The DNA data storage system of claim 1, comprising at least 2 and no more than 10 distinct synthetic nucleotides.

9. The DNA data storage system of claim 1, comprising 7 distinct synthetic nucleotides.

10. A method of reading a DNA sequence, the method comprising:
introducing a DNA data storage system into a flow cell of a nanopore sequencing device, wherein the DNA data storage system comprises a modification region comprising synthetic nucleotides;
receiving information indicative of an electrical signal provided when the modification region passes through a nanopore of the nanopore sequencing device;
classifying, based on the received information, at least a portion of the modification region according to an expanded molecular alphabet; and
determining, based on the classifying, a nucleotide sequence of the modification region.

11. The method of claim 10, wherein the DNA data storage system further comprises a calibration region, wherein the method further comprises:
determining calibration information corresponding to the calibration region;
calibrating the nanopore sequencing device based on the calibration information, wherein the calibrating compensates for level drift.

12. The method of claim 10 or claim 11, wherein the classifying is performed using a trained neural network.

13. The method of claim 12, wherein the trained neural network comprises a convolutional neural network.

14. The method of claim 12, wherein the trained neural network comprises a 1-dimensional residual neural network.

15. The method of claim 14, wherein the 1-dimensional residual neural network comprises:
a plurality of 1-dimensional convolution layers; and
a fully connected layer, wherein the fully-connected layer is configured to perform the classifying step.

16. The method of claim 15, wherein at least a portion of the 1-dimensional convolution layers comprise a kernel size of 1 by 8.

17. The method of claim 15, wherein the trained neural network comprises a plurality of output channels.

18. The method of claim 15, wherein the plurality of output channels comprises 64 output channels.

19. The method of claim 15, wherein the plurality of 1-dimensional convolution layers comprises nine 1-dimensional convolution blocks, wherein the 1-dimensional convolution layers are configured to perform feature extraction from the received information.

20. A method of training a neural network comprising:
providing training data to the neural network, wherein the training data comprises labeled data, wherein the labeled data comprises values indicative of electrical signals provided when a modification region of a DNA data storage system passes through a nanopore of a nanopore sequencing device, wherein the labeled data further comprises labels corresponding to an expanded molecular alphabet; and
comparing an output of the neural network to the labels;
adjusting at least one weight of the neural network based on the comparison.
